(19) United States
(12) Patent Application Publication
(10) Pub. No.: US 2019/0365822 A1
(43) Pub. Date: Dec. 5, 2019

(54) CARDIAC PROGENITOR CELLS HAVING ENHANCED p53 EXPRESSION AND USES THEREOF

(71) Applicant: AAL SCIENTIFICS, INC., New York, NY (US)

(72) Inventors: Piero ANVERSA, New York, NY (US); Annarosa LERI, New York, NY (US)

(73) Assignee: AAL SCIENTIFICS, INC., New York, NY (US)

(21) Appl. No.: 16/481,617

(22) PCT Filed: Feb. 1, 2018
(86) PCT No.: PCT/US2018/016372

§ 371 Date: Jul. 29, 2019

Related U.S. Application Data
(60) Provisional application No. 62/453,421, filed on Feb. 1, 2017.

(51) Int. Cl. A61K 35/34; C12N 5/077; A61P 9/00

(52) U.S. Cl. CPC A61K 35/34; A61P 9/00; C12N 5/0657

(57) ABSTRACT

Disclosed herein are compositions comprising cardiac progenitor cells that express exogenous p53 protein. Such compositions are useful for treating cardiac diseases or disorders. Also disclosed herein are methods of producing cardiac progenitor cells that express exogenous p53.

This application claims priority to and benefit of U.S. Provisional Patent Application No. 62/453,421, filed on Feb. 1, 2017. The contents of this application are herein incorporated by reference in their entirety.

### STATEMENT OF GOVERNMENT INTEREST

This invention was made with government support under Grant No. NIA/R01AG37490 awarded by the National Institutes of Health. The government has certain rights in the invention.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: AALS_007_01 WO_SeqList_ST25.txt; date recorded: Feb. 1, 2018; file size 3,745 bytes).

### FIELD OF THE INVENTION

The present invention relates generally to the field of cardiology. More specifically, the invention relates to cardiac progenitor cells that express exogenous p53 protein and the use of such cells to treat or prevent heart diseases or disorders.

### BACKGROUND OF THE INVENTION

Myocardial aging in animals and humans is characterized by an increase in number of resident cardiac progenitor cells (CPCs) expressing the senescence-associated protein p16^{INK4a}, which prevents permanently the reentry of stem cells into the cell cycle (Beausejour and Campisi, 2006, Dimmeler and Leri, 2008, Sanada et al., 2014, Leri et al., 2015). This age-dependent effect results in a reduction of the pool of functionally-competent CPCs in the old heart (Torella et al., 2004). Alterations of coronary blood flow and defects in the structural determinants of tissue oxygenation in the aging myocardium (Hachamovitch et al., 1989) create hypoxic micro-domains where CPCs are maintained in a quiescent state (Sanada et al., 2014), impairing the activation of a compartment of progenitor cells with relatively intact replicative reserve.

Ongoing clinical trials with autologous cardiac stem cells (CSCs) are faced with a critical limitation which is related to the modest amount of retained cells within the damaged myocardium. There is a need for compositions and methods that can be used to restore the structural and functional integrity of the decompensated heart.

### SUMMARY OF THE INVENTION

In one embodiment, provided herein is a method of treating or preventing a heart disease or disorder in a subject in need thereof comprising administering isolated cardiac progenitor cells (CPCs) to the subject, wherein the CPCs comprise one or more copies of a tumor suppressor p53 gene in addition to the endogenous copy of a p53 gene. In some embodiments, the heart disease or disorder is heart failure, diabetic heart disease, rheumatic heart disease, hypertensive heart disease, ischemic heart disease, cerebrovascular heart disease, inflammatory heart disease and/or congenital heart disease. In some embodiments, the CPCs express an increased amount of p53 protein compared to the amount expressed by CPCs that do not comprise one or more copies of a p53 gene in addition to the endogenous copy of a p53 gene.

In one embodiment, the invention provides a method of repairing and/or regenerating damaged tissue of a heart in a subject in need thereof comprising: (a) extracting cardiac progenitor cells (CPCs) from a heart; (b) introducing one or more tumor suppressor p53 genes into the CPCs of step (a); (c) culturing and expanding said CPCs from step (b); and (d) administering a dose of said CPCs from step (c) to an area of damaged tissue in the subject effective to repair and/or regenerate the damaged tissue of the heart. In some cases, the subject has diabetes.

In one embodiment, the invention provides a method of promoting cellular engraftment and growth of cardiac progenitor cells (CPCs) in damaged tissue of a heart in a subject in need thereof comprising: (a) extracting cardiac progenitor cells (CPCs) from a heart; (b) introducing one or more tumor suppressor p53 genes into the CPCs of step (a); (c) culturing and expanding said CPCs from step (b); and (d) administering a dose of said CPCs from step (c) to an area of damaged tissue in the subject effective to promote cellular engraftment and growth of the CPCs in the damaged tissue of the heart in a subject in need thereof. In some cases, the subject has diabetes.

The invention further provides a method of producing a large quantity of cardiac progenitor cells (CPCs) comprising: (a) isolating CPCs from heart tissue: (b) introducing one or more tumor suppressor p53 genes into the CPCs of step (a); and (c) culturing and expanding the CPCs of step (b), thereby producing a large quantity of CPCs.

In one embodiment, the invention provides a method of promoting cellular engraftment and growth of cells in an organ or tissue during cell therapy, comprising: (a) extracting cells from an organ or tissue; (b) introducing one or more tumor suppressor p53 genes into the cells of step (a); (c) culturing and expanding said cells from step (b); and (d) applying an amount of said cells from step (c) to an area of damaged organ or tissue, thereby promoting cellular engraftment and growth of cells in the damaged organ or tissue.

In one embodiment, the invention provides a method of producing isolated cardiac progenitor cells (CPCs) having an improved ability to tolerate oxidative stress, comprising: (a) isolating CPCs from heart tissue; (b) introducing one or more tumor suppressor p53 genes into the CPCs of step (a); and (c) culturing and expanding the CPCs of step (b), thereby producing CPCs having an improved ability to tolerate oxidative stress compared to CPCs from step (a).

In one embodiment, the invention provides a method of producing isolated cardiac progenitor cells (CPCs) having restored DNA integrity, comprising: (a) isolating CPCs from heart tissue; (b) introducing one or more tumor suppressor p53 genes into the CPCs of step (a); and (c) culturing and expanding the CPCs of step (b), thereby producing CPCs having restored DNA integrity compared to CPCs from step (a).

In one embodiment, the invention provides a method of producing isolated cardiac progenitor cells (CPCs) having an improved proliferative capacity, comprising: (a) isolating CPCs from heart tissue; (b) introducing one or more tumor suppressor p53 genes into the CPCs of step (a); and (c) culturing and expanding the CPCs of step (b), thereby producing CPCs having an improved proliferative capacity compared to CPCs from step (a).

In one embodiment, the invention provides a pharmaceutical composition comprising a therapeutically effective amount of isolated cardiac progenitor cells (CPCs) and a pharmaceutically acceptable carrier for repairing and/or regenerating damaged tissue of a heart, wherein said isolated CPCs comprise one or more copies of a tumor suppressor p53 gene in addition to the endogenous copy of a p53 gene.

In another embodiment, the invention provides a pharmaceutical composition comprising a therapeutically effective amount of isolated cardiac progenitor cells (CPCs) and a pharmaceutically acceptable carrier for promoting cellular engraftment and growth of the CPCs in damaged tissue of a heart, wherein said isolated CPCs comprise one or more copies of a tumor suppressor p53 gene in addition to the endogenous copy of a p53 gene.

In one embodiment, the invention provides a pharmaceutical composition comprising a therapeutically effective amount of cells and a pharmaceutically acceptable carrier for promoting cellular engraftment and growth of the cells in a damaged organ or tissue, wherein said cells comprise one or more copies of a tumor suppressor p53 gene in addition to the endogenous copy of a p53 gene.

### BRIEF DESCRIPTION OF THE DRAWINGS

FIGS. 1A-1C depict results showing that aging and p53 do not alter cardiac and myocyte function. (FIG. 1A) Hemodynamics in young-adult (3-6 months) and old (24-31 months) p53-tg and WT mice (young WT, n=9, young p53-tg, n=7; old WT, n=11, old p53-tg, n=6). LV SP, LV systolic pressure; LV EndDP, LV end-diastolic pressure; LV DevP, LV developed pressure. (FIG. 1B) Ca²⁺ transients and sarcomere shortening of cardiomyocytes in young WT (n=112 cells from 10 mice) and young p53-tg (n=79 cells from ±7 mice). (FIG. 1C) Ca²⁺ transients and sarcomere shortening of cardiomyocytes in old WT (n=40 cells from 3 mice) and old p53-tg (n=25 cells from 3 mice). “LV” refers to “left ventricle”.

FIGS. 2A-2I depict characterization of p53, cardiomyocytes and CPCs in WT and p53-tg mice. (FIGS. 2A-2B) Ki67-positive (FIG. 2A) and apoptotic TUNEL-positive (FIG. 2B) cardiomyocytes in young-adult, 8-11 months (WT: n=9; p53-tg: n=7), and old, 20-25 months (WT: n=6; p53-tg: n=8), WT and p53-tg mice. *p<0.05 vs. young-adult WT; **p<0.05 vs. old WT; ***p<0.05 vs. young-adult p53-tg. (FIG. 2C) p16^{INK4a}-positive cardiomyocytes in old, 18-33 months, WT (n=4) and p53-tg (n=9) mice. (FIGS. 2D-2E) Number of c-kit-positive CPCs in atrial myocardium (FIG. 2D) and fraction of cycling Ki67-positive CPCs (FIG. 2E). WT: n=3; p53-tg: n=4. (FIG. 2F) Population doubling time (PDT) in WT-CPCs (WT; n=3) and p53-tg-CPCs (p53-tg; n=3). (FIG. 2G) Fraction of Ki67 labeled WT-CPCs (n=3) and p53-tg-CPCs (n=3). (FIG. 2H) Fraction of p16^{INK4a} labeled WT-CPCs (n=3) and p53-tg-CPCs (n=3). (FIG. 2I) Apoptosis of WT-CPCs (n=3) and p53-tg-CPCs (n=3) measured by Annexin V assay. In all cases data are shown as mean±SD. *p<0.05 vs. WT.

FIGS. 3A-3F show that p53 improves the DDR of CPCs. (a) Nuclei from p53-tg-CPCs in the absence (Control) and in the presence of doxorubicin (Doxo) are stained by DAPI (blue, left panels); immunolabeled γH2A.X is shown in these nuclei (green, right panels). Scale bar: 100 μm. (b) Fraction of γH2A.X-positive CPCs in the absence (control, Ctrl) and following exposure to Doxo (Doxo): Ctrl WT-CPCs (4284 cells from 3 mice); Ctrl p53-tg-CPCs (13,334 cells from 3 mice); Doxo WT-CPCs (3958 cells from 3 mice); and Doxo p53-tg-CPCs (16,496 cells from 3 mice). Data are mean±SD. (c) γH2A.X (green; left two panels) in nuclei of WT-CPCs and p53-tg-CPCs stained by DAPI (blue). DDR foci are illustrated in the same nuclei following three-dimensional reconstruction by Imaris version 5.5.2 (right two panels). Scale bar: 5 μm. (d) Number of DDR foci counted in nuclei of WT-CPCs and p53-tg-CPCs. In each case, 24-59 γH2A.X positive nuclei from 3 mice were analyzed. (e) Nucleoids of WT-CPCs and p53-tg-CPCs are stained with Vista green dye (green, left panels). Comets are apparent after Doxo (green, right panels). (f) Quantity of damaged DNA in nuclei of WT-CPCs and p53-tg-CPCs at baseline (Control: WT, n=62 comets from 3 mice; p53-tg, n=70 comets from 3 mice) and after Doxo (Doxo: WT, n=76 comets from 3 mice; p53-tg, n=61 comets from 3 mice). *p<0.05 vs. WT Ctrl; **p<0.05 vs. Doxo WT-CPCs; ***p<0.05 vs. p53-tg Ctrl.

FIGS. 4A-4D depict the expression of p53 and p53-dependent genes. (a) Quantity of p53 protein by automated Wes Western blotting in WT-CPCs (WT) and p53-tg-CPCs (p53-tg) at baseline (blue line) and after Doxo (red line). Tracings illustrate the peak level of p53 in the four CPC classes; n=3 in all cases. (b) The pseudo-blots show the expression of phosphorylated p53 at Ser-18 and Ser-34, and p53 and GAPDH in the four CPC classes. (c) Quantitative data are shown as mean±SD. *p<0.05 vs. WT Ctrl. **p<0.05 vs. WT Doxo. ***p<0.05 vs. p53-tg Ctrl. (d) mRNA level of p53 and p53 regulated genes in the CPC classes at baseline (Ctrl) and after Doxo; n=3 in all cases. Ct values above 35 cycles were considered not detectable. For statistics see panel B.

FIGS. 5A-5F depict that p53 favors the functional recovery of CPCs from oxidative stress in vitro. (a) Western blotting of p16^{INK4a} at baseline, after Doxo-pulse and following recovery of WT-CPCs (WT) and p53-tg-CPCs (p53-tg); n=3 in all cases. Optical density data are mean±SD. *p<0.05 vs. WT-Control. **p<0.05 vs. WT-Doxo-pulse. ***p<0.05 vs. WT-recovery. (b) p16^{INK4a} labeling (upper left panel, yellow) of WT-CPCs exposed to Doxo. Nuclei are stained by DAPI (upper right panel, blue). Phalloidin (lower left panel, white). Merge of p16^{INK4a}, DAPI and phalloidin (lower right panel). Scale bar, 50 μm. Fraction of p16^{INK4a}-positive WT-CPCs and p53-tg-CPCs at baseline, following Doxo-pulse and after recovery; n=3 in all cases. Data are mean±SD. *p<0.05 vs. WT-Control. **p<0.05 vs. WT-Doxo-pulse. ***p<0.05 vs. WT recovery. ^{†p<}0.05 vs. p53-tg control. ^{‡}p<0.05 vs. p53-tg Doxo-pulse. (c) Number of DDR foci in WT-CPCs and p53-tg-CPCs at baseline, after Doxo-pulse and following recovery; n=3 in all cases. For statistics see panel B. (d) Nucleoids in WT-CPCs and p53-tg-CPCs at baseline, following Doxo-pulse and after recovery are stained with Vista green dye (green). Comets are apparent in Doxo-pulse and after recovery of WT-CPCs, while intact DNA is noted in p53-tg-CPCs after recovery. (e) Damaged DNA in nuclei of WT-CPCs and p53-tg-CPCs at baseline, after Doxo-pulse and following recovery; n=3 in all cases. For statistics see panel B. (f) Fraction of Ki67-positive WT-CPCs and p53-tg-CPCs following 24, 48 and 72 h recovery period; n=3 in all cases. *p<0.05 vs. 24 h. **p<0.05 vs. 48 h.

FIGS. 6A-6B depict that p53-tg-CPCs engraft in the diabetic heart. (FIGS. 6A-6B) Areas of myocardial damage (*) in the LV wall; EGFP-positive (green) p53-tg-CPCs are engrafted in the majority of these foci of injury. Cardiomyocytes are labeled by α-sarcomeric actin (α-SA; red).

FIGS. 7A-7E depict that p53 expands the engraftment of CPCs within the diabetic myocardium. (FIGS. 7A-7D) Areas of myocardial regeneration shown at different magnification contain small developing cardiomyocytes, which express EGFP and α-SA (yellow; arrows). (FIG. 7E) Number of EGFP-positive cells per 10 mm² of myocardium in diabetic hearts injected with WT-CPCs (n=4) or p53-tg-CPCs (n=4). Data are mean±SD. *p<0.05 vs. WT-CPCs.

FIGS. 8A-8C depict the early commitment of p53-tg-CPCs. (FIGS. 8A-8C) GATA4 is expressed (left, white) in EGFP-positive cells (right, green) distributed within the damaged diabetic myocardium. Cardiomyocytes are labeled by troponin I (right, TnI: red).

FIGS. 9A-9D depict the expression of p53 and p53 target genes. (a-d) Expression of Bcl2 (FIG. 9A), Bax (FIG. 9B), Aogen (FIG. 9C) and AT1R (FIG. 9D) in cardiomyocytes of WT (n=4-5) and p53-tg (n=5-7). Loading conditions were established by Ponceau red, which was employed for normalization of protein expression. A non-specific band is located above 26 kDa in the Bcl2 blot.

FIG. 10 depicts the expression of p53 and p53-dependent genes. Time-dependent changes in the expression of p53 and p53-related genes in p53-tg-CPCs (green line) and WT-CPCs (red line) following exposure to Doxo; n=3 in all cases.

FIGS. 11A-1D depict CPCs and the diabetic heart. (FIGS. 11A-11D) Areas of myocardial damage (*) in the LV wall; EGFP-positive (green) WT-CPCs are engrafted in some of these foci of injury.

FIGS. 12A-12B depict the early commitment of WT-CPCs. (FIGS. 12A-12B) GATA4 is expressed (left, white) in EGFP-positive cells (right, green) distributed within the damaged diabetic myocardium. Cardiomyocytes are labeled by troponin I (right, TnI: red).

FIGS. 13A-13C depict p53 and p53-dependent genes and their function. DNA damage activates pathways resulting in the inhibition of cell growth and apoptosis, or DNA repair and proliferation. Red arrows, WT; green arrows, p53-tg.

### DETAILED DESCRIPTION OF THE INVENTION

The invention described herein is based on the discovery that cardiac progenitor cells (CPCs) with enhanced expression of tumor suppressor p53 are useful for therapeutic purposes. Ongoing clinical trials with autologous cardiac stem cells (CSCs) are faced with a critical problem which is related to the modest amount of retained cells within the damaged myocardium. Provided herein is a strategy that overcomes in part this problem by enhancing the number of CSCs able to engraft within the pathologic organ. Additionally, these genetically modified CSCs can be generated in large number, raising the possibility that multiple temporally distinct deliveries of cells can be introduced to restore the structural and functional integrity of the decompensated heart.

p53 is an important modulator of stem cell fate, but its role in cardiac progenitor cells (CPCs) was unknown. An amino acid sequence of human p53 may be found at GenBank™ Accession No. BAC16799.1. The effects of a single extra-copy of p53 on the function of CPCs in the presence of oxidative stress mediated by doxorubicin in vitro and type-1 diabetes in vivo were tested. CPCs were obtained from super-p53 transgenic mice (p53-tg), in which the additional allele is regulated in a manner similar to the endogenous protein. Old CPCs with increased p53 dosage showed a superior ability to sustain oxidative stress, repair DNA damage and restore cell division. With doxorubicin, a larger fraction of CPCs carrying an extra-copy of the p53 allele recruited γH2A.X reestablishing DNA integrity. Enhanced p53 expression resulted in a superior tolerance to oxidative stress in vivo by providing CPCs with defense mechanisms necessary to survive in the milieu of the diabetic heart; they engrafted in regions of tissue injury and in three days acquired the cardiomyocyte phenotype. This genetic strategy of increased dosage of p53 in CPCs can be translated to humans to increase cellular engraftment and growth, critical determinants of successful cell therapy for the failing heart.

The tumor suppressor p53 is a major regulator of DNA repair and cell division, cellular aging and apoptosis (Riley et al., 2008). Phosphorylation of the N-terminal of p53 promotes DNA repair, a process that is intimately linked to the progression of the cell cycle. DNA repair may be less effective in old CPCs, resulting in the accumulation of DNA lesions, a phenomenon that favors cellular senescence. The expression of p53 increases with aging and heart failure (Leri et al., 2003, Cheng et al., 2013) but its actual role in CPCs is unknown; p53 may trigger apoptosis of old cells and may induce DNA repair in cells with a younger phenotype (Matheu et al., 2007).

Whether this potential youth promoting effect of p53 is determined by a successful DNA damage response (DDR), mediated by transient reparable DNA lesions in the telomeric and non-telomeric regions of the genome, has not been defined. A prolonged DDR signaling may result in the accumulation of non-reparable DNA foci and initiation of cell death (Fumagalli et al., 2012). Moreover, these intrinsic variables of CPCs have implications in the outcome of cell therapy for the damaged heart, where the unfavorable conditions of the recipient myocardium with high levels of oxidative stress affect the survival and growth of the delivered cells. These questions were addressed herein by evaluating CPC aging in mice with enhanced expression of p53 and then by assessing CPC engraftment in the diabetic heart that is characterized by an environment in which the generation of reactive oxygen and inflammation condition its evolution (Rota et al., 2006).

The super-p53 mouse (p53-tg) (Garcia-Cao et al., 2002), which is based on a C57BL/6J genetic background, carries a single extra gene-dose of p53. This single-copy transgene is regulated in a manner similar to its endogenous counterpart; p53 is not constitutively active, but undergoes post-translational modifications in response to stress stimuli, resulting in a moderately higher p53 activity (Garcia-Cao et al., 2006). The increased gene dosage of p53 triggers an amplified DDR in lymphocytes, splenocytes, embryonic fibroblasts, and epithelial cells of the skin and intestine (Garcia-Cao et al., 2002), but its impact on CPC aging and growth reserve has never been determined previously. Because of these characteristics, this animal model was considered relevant for understanding the role of p53 in CPC function with aging and oxidative stress.

In some embodiments, the invention provides a recombinant CPC (or a plurality of CPCs) comprising one or more copies of a tumor suppressor p53 gene in addition to the endogenous copy of a p53 gene. In some embodiments, a recombinant CPC comprises one, two or three copies of a tumor suppressor p53 gene in addition to the endogenous copy of a p53 gene. In some embodiments, recombinant CPCs of the invention express an increased amount of p53 protein or p53 mRNA compared to the amount expressed by an equivalent number of CPCs (also referred to as wild-type (WT) CPCs) that do not comprise one or more copies of a p53 gene in addition to the endogenous copy of a p53 gene. Amounts of p53 protein or p53 mRNA may be measured by standard assays known in the art. For example, western blot, ELISA, northern blot or quantitative PCR may be used. In some embodiments, recombinant CPCs of the invention express at least about 10%, 20%, 300%, 40%, 50%, 60%, 70%, 80%, 90% or 100% more p53 protein or p53 mRNA compared to the amount expressed by an equivalent number of WT CPCs. In some embodiments, recombinant CPCs of the invention express at least about 2-fold, 3-fold, 4-fold, 5-fold, 6-fold, 7-fold, 8-fold, 9-fold or 10-fold more p53 protein or p53 mRNA compared to the amount expressed by an equivalent number of WT CPCs. In some embodiments, recombinant CPCs of the invention have enhanced expression of tumor suppressor p53.

In some embodiments, the recombinant CPCs comprising one, two or three copies of a tumor suppressor p53 gene in addition to the endogenous copy of a p53 gene have an improved ability to tolerate oxidative stress compared to WT CPCs. In some embodiments, the recombinant CPCs of the invention have restored DNA integrity compared to WT CPCs. In some embodiments, the recombinant CPCs of the invention have an improved proliferative capacity compared to WT CPCs.

In one embodiment, the invention provides a pharmaceutical composition comprising a therapeutically effective amount of isolated cardiac progenitor cells (CPCs) and a pharmaceutically acceptable carrier for repairing and/or regenerating damaged tissue of a heart, wherein said isolated CPCs comprise one or more copies of a tumor suppressor p53 gene in addition to the endogenous copy of a p53 gene.

In another embodiment, the invention provides a pharmaceutical composition comprising a therapeutically effective amount of isolated cardiac progenitor cells (CPCs) and a pharmaceutically acceptable carrier for promoting cellular engraftment and growth of the CPCs in damaged tissue of a heart, wherein said isolated CPCs comprise one or more copies of a tumor suppressor p53 gene in addition to the endogenous copy of a p53 gene.

In one embodiment, the invention provides a pharmaceutical composition comprising a therapeutically effective amount of cells and a pharmaceutically acceptable carrier for promoting cellular engraftment and growth of the cells in a damaged organ or tissue, wherein said cells comprise one or more copies of a tumor suppressor p53 gene in addition to the endogenous copy of a p53 gene.

When recombinant CPCs comprising one, two or three copies of a tumor suppressor p53 gene in addition to the endogenous copy of a p53 gene are placed into a mouse with a damaged heart, long-term engraftment of the administered CPCs occurs, and these CPCs differentiate into, for example, cardiomyocytes, which can lead to subsequent heart tissue regeneration and repair. The mouse experiments indicate that isolated recombinant CPCs described herein can be used for heart tissue regeneration in human patients (e.g., diabetic human patients). Accordingly, provided herein are methods for the treatment and/or prevention of a heart disease or disorder in a subject in need thereof. In some embodiments, provided herein is a method of treating or preventing a heart disease or disorder in a subject in need thereof, comprising administering isolated cardiac progenitor cells (CPCs) to the subject, wherein the CPCs comprise one or more copies of a tumor suppressor p53 gene in addition to the endogenous copy of a p53 gene.

In some embodiments, a subject treated by the methods and compositions described herein has a heart disease or disorder. As used herein, the term “heart disease or disorder”, “heart disease”, “heart condition” and “heart disorder” are used interchangeably. Heart diseases and/or conditions can include heart failure, diabetic heart disease, rheumatic heart disease, hypertensive heart disease, ischemic heart disease, cerebrovascular heart disease, inflammatory heart disease and/or congenital heart disease. The methods described herein can be used to treat, ameliorate the symptoms, prevent and/or slow the progression of a number of heart diseases or disorders or their symptoms. In some embodiments of all aspects of the therapeutic methods described herein, a subject having a heart disease or disorder is first selected prior to administration of the recombinant CPCs.

In some embodiments, recombinant CPCs comprising one, two or three copies of a tumor suppressor p53 gene in addition to the endogenous copy of a p53 gene can repair damaged heart tissue in diabetic mice. Examples of mouse models of diabetes and methods of implanting stem cells in such mice are described in e.g., Hua et al., PLoS One, 2014 Jul. 10; 9(7):e102198. In some embodiments, provided herein is a method of treating or preventing a heart disease or disorder in a diabetic subject in need thereof, comprising administering isolated cardiac progenitor cells (CPCs) to the subject, wherein the CPCs comprise one or more copies of a tumor suppressor p53 gene in addition to the endogenous copy of a p53 gene. In some embodiments, a subject treated by the methods or compositions described herein has type 1 diabetes or type 2 diabetes.

In one embodiment, the invention provides a method of repairing and/or regenerating damaged tissue of a heart in a subject in need thereof comprising: (a) extracting cardiac progenitor cells (CPCs) from a heart; (b) introducing one or more tumor suppressor p53 genes into the CPCs of step (a); (c) culturing and expanding said CPCs from step (b); and (d) administering a dose of said CPCs from step (c) to an area of damaged tissue in the subject effective to repair and/or regenerate the damaged tissue of the heart.

In one embodiment, the invention provides a method of promoting cellular engraftment and growth of cardiac progenitor cells (CPCs) in damaged tissue of a heart in a subject in need thereof comprising: (a) extracting cardiac progenitor cells (CPCs) from a heart; (b) introducing one or more tumor suppressor p53 genes into the CPCs of step (a); (c) culturing and expanding said CPCs from step (b); and (d) administering a dose of said CPCs from step (c) to an area of damaged tissue in the subject effective to promote cellular engraftment and growth of the CPCs in the damaged tissue of the heart in a subject in need thereof.

The terms “subject”, “patient” and “individual” are used interchangeably herein, and refer to an animal, for example, a human from whom cells for use in the methods described herein can be obtained (i.e., donor subject) and/or to whom treatment, including prophylactic treatment, with the cells as described herein, is provided, i.e., recipient subject. For treatment of those conditions or disease states that are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or food production mammal, e.g., cow, sheep, pig, and the like.

Accordingly, in some embodiments of the therapeutic methods described herein, a subject is a recipient subject, i.e., a subject to whom the recombinant CPCs described herein are being administered, or a donor subject, i.e., a subject (e.g., a mouse) from whom a heart tissue sample comprising recombinant CPCs described herein is being obtained. A recipient or donor subject can be of any age. In some embodiments, the subject is a “young subject,” defined herein as a subject less than 10 years of age. In other embodiments, the subject is an “infant subject,” defined herein as a subject is less than 2 years of age. In some embodiments, the subject is a “newborn subject,” defined herein as a subject less than 28 days of age. In one embodiment, the subject is a human adult.

The isolated recombinant CPCs described herein can be administered to a selected subject having any heart disease or disorder or predisposed to developing a heart disease or disorder. The administration can be by any appropriate route which results in an effective treatment in the subject. In some aspects of these methods, a therapeutically effective amount of isolated recombinant CPCs described herein is administered through vessels, directly to the tissue, or a combination thereof. Some of these methods involve administering to a subject a therapeutically effective amount of isolated recombinant CPCs described herein by injection, by a catheter system, or a combination thereof.

As used herein, the terms “administering,” “introducing”, “transplanting” and “implanting” are used interchangeably in the context of the placement of cells, e.g., recombinant CPCs of the invention into a subject, by a method or route which results in at least partial localization of the introduced cells at a desired site, such as a site of injury or repair, such that a desired effect(s) is produced. The cells, e.g., recombinant CPCs, or their differentiated progeny (e.g., cardiomyocytes) can be implanted directly to the heart, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. For example, in some embodiments of all aspects of the therapeutic methods described herein, an effective amount of a population of isolated recombinant CPCs is administered directly to the heart of an individual suffering from heart disease by direct injection. In other embodiments of all aspects of the therapeutic methods described herein, the population of isolated recombinant CPCs is administered via an indirect systemic route of administration, such as a catheter-mediated route.

One embodiment of the invention includes use of a catheter-based approach to deliver the injection. The use of a catheter precludes more invasive methods of delivery such as surgically opening the body to access the heart. As one skilled in the art is aware, optimum time of recovery would be allowed by the more minimally invasive procedure, which as outlined here, includes a catheter approach. When provided prophylactically, the isolated recombinant CPCs can be administered to a subject in advance of any symptom of a heart disease or disorder. Accordingly, the prophylactic administration of an isolated recombinant CPCs population serves to prevent a heart disease or disorder, or further progress of heart diseases or disorders as disclosed herein.

When provided therapeutically, isolated recombinant CPCs are provided at (or after) the onset of a symptom or indication of a heart disease or disorder, or for example, upon the onset of diabetes.

As used herein, the terms “treat,” “treatment,” “treating,” or “amelioration” refer to therapeutic treatment, wherein the object is to reverse, alleviate, ameliorate, decrease, inhibit, or slow down the progression or severity of a condition associated with a disease or disorder. The term “treating” includes reducing or alleviating at least one adverse effect or symptom of a condition, disease or disorder associated with a heart disease). Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. In some embodiments, “treatment” and “treating” can also mean prolonging survival of a subject as compared to expected survival if the subject did not receive treatment.

As used herein, the term “prevention” refers to prophylactic or preventative measures wherein the object is to prevent or delay the onset of a disease or disorder, or delay the onset of symptoms associated with a disease or disorder. In some embodiments, “prevention” refers to slowing down the progression or severity of a condition or the deterioration of cardiac function associated with a heart disease or disorder.

In another embodiment, “treatment” of a heart disease or disorder also includes providing relief from the symptoms or side-effects of the disease (including palliative treatment). In some embodiments of the aspects described herein, the symptoms or a measured parameter of a disease or disorder are alleviated by at least 5%, at least 10%, at least 20%, at least 30%, at least 40%, at least 50%, at least 60%, at least 70%, at least 80%, or at least 90%, upon administration of a population of isolated recombinant CPCs, as compared to a control or non-treated subject.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions as disclosed herein will be decided by the attending physician within the scope of sound medical judgment. The exact amount required will vary depending on factors such as the type of heart disease or disorder being treated, degree of damage, whether the goal is treatment or prevention or both, age of the subject, the amount of cells available, etc. Thus, one of skill in the art realizes that a treatment may improve the disease condition, but may not be a complete cure for the disease.

In one embodiment of all aspects of the therapeutic methods described, the term “effective amount” as used herein refers to the amount of a population of isolated recombinant CPCs needed to alleviate at least one or more symptoms of the heart disease or disorder, and relates to a sufficient amount of pharmacological composition to provide the desired effect, e.g., treat a subject having heart disease. The term “therapeutically effective amount” therefore refers to an amount of isolated recombinant CPCs using the therapeutic methods as disclosed herein that is sufficient to effect a particular effect when administered to a typical subject, such as one who has or is at risk for heart disease.

In another embodiment of all aspects of the methods described, an effective amount as used herein would also include an amount sufficient to prevent or delay the development of a symptom of the disease, alter the course of a disease symptom (for example, but not limited to, slow the progression of a symptom of the disease), or even reverse a symptom of the disease. The effective amount of recombinant CPCs needed for a particular effect will vary with each individual and will also vary with the type of heart disease or disorder being addressed. Thus, it is not possible to specify the exact “effective amount”. However, for any given case, an appropriate “effective amount” can be determined by one of ordinary skill in the art using routine experimentation.

In some embodiments of all aspects of the therapeutic methods described, the subject is first diagnosed as having a disease or disorder affecting the heart prior to administering the recombinant CPCs according to the methods described herein. In some embodiments of all aspects of the therapeutic methods described, the subject is first diagnosed as being at risk of developing a heart disease or disorder prior to administering the recombinant CPCs, e.g., an individual with a genetic disposition for heart disease or diabetes or who has close relatives with heart disease or diabetes.

For use in all aspects of the therapeutic methods described herein, an effective amount of isolated recombinant CPCs comprises at least 10², at least 5×10², at least 10³, at least 5×10′, at least 10⁴, at least 5×10⁴, at least 10⁵, at least 2×10⁵, at least 3×10⁵, at least 4×10⁵, at least 5×10⁵, at least 6×10⁵, at least 7×10⁵, at least 8×10⁵, at least 9×10⁵, or at least 1×10⁶ recombinant CPCs or multiples thereof per administration. In some embodiments, more than one administration of isolated recombinant CPCs is performed to a subject. The multiple administration of isolated recombinant CPCs can take place over a period of time. The recombinant CPCs can be generated from CPCs isolated from one or more donors, or from CPCs obtained from an autologous source.

Exemplary modes of administration of recombinant CPCs and other agents for use in the methods described herein include, but are not limited to, injection, infusion, inhalation (including intranasal), ingestion, and rectal administration. “Injection” includes, without limitation, intravenous, intraarterial, intraductal, direct injection into the tissue intraventricular, intracardiac, transtracheal injection and infusion. The phrases “parenteral administration” and “administered parenterally” as used herein, refer to modes of administration other than enteral and topical administration, usually by injection, and includes, without limitation, intravenous, intraventricular, intracardiac, transtracheal injection and infusion. In some embodiments, recombinant CPCs can be administered by intravenous, intraarterial, intraductal, or direct injection into tissue, or through injection in the liver.

In some embodiments of all aspects of the therapeutic methods described, an effective amount of isolated recombinant CPCs is administered to a subject by injection. In other embodiments, an effective amount of isolated recombinant CPCs is administered to a subject by a catheter-mediated system. In other embodiments, an effective amount of isolated recombinant CPCs is administered to a subject through vessels, directly to the tissue, or a combination thereof. In additional embodiments, an effective amount of isolated recombinant CPCs is implanted in a patient in an encapsulating device (see, e.g., U.S. Pat. Nos. 9,132,226 and 8,425,928, the contents of each of which are incorporated herein by reference in their entirety).

In some embodiments of all aspects of the therapeutic methods described, an effective amount of isolated recombinant CPCs is administered to a subject by systemic administration, such as intravenous administration.

The phrases “systemic administration,” “administered systemically”, “peripheral administration” and “administered peripherally” as used herein refer to the administration of population of recombinant CPCs other than directly into the heart, such that it enters, instead, the subject's circulatory system.

In some embodiments of all aspects of the therapeutic methods described, one or more routes of administration are used in a subject to achieve distinct effects. For example, isolated recombinant CPCs are administered to a subject by both direct injection and catheter-mediated routes for treating or repairing heart tissue. In such embodiments, different effective amounts of the isolated recombinant CPCs can be used for each administration route.

In some embodiments of all aspects of the therapeutic methods described, the methods further comprise administration of one or more therapeutic agents, such as a drug or a molecule, that can enhance or potentiate the effects mediated by the administration of the isolated recombinant CPCs, such as enhancing homing or engraftment of the recombinant CPCs, increasing repair of cardiac cells, or increasing growth and regeneration of cardiac cells. The therapeutic agent can be a protein (such as an antibody or antigen-binding fragment), a peptide, a polynucleotide, an aptamer, a virus, a small molecule, a chemical compound, a cell, a drug, etc.

As defined herein, “vascular regeneration” refers to de novo formation of new blood vessels or the replacement of damaged blood vessels (e.g., capillaries) after injuries or traumas, as described herein, including but not limited to, heart disease. “Angiogenesis” is a term that can be used interchangeably to describe such phenomena.

In some embodiments of all aspects of the therapeutic methods described, the methods further comprise administration of recombinant CPCs together with growth, differentiation, and angiogenesis agents or factors that are known in the art to stimulate cell growth, differentiation, and angiogenesis in the heart tissue. In some embodiments, any one of these factors can be delivered prior to or after administering the compositions described herein. Multiple subsequent delivery of any one of these factors can also occur to induce and/or enhance the engraftment, differentiation and/or angiogenesis. Suitable growth factors include but are not limited to ephrins (e.g., ephrin A or ephrin B), transforming growth factor-beta (TGFβ), vascular endothelial growth factor (VEGF), platelet derived growth factor (PDGF), angiopoietins, epidermal growth factor (EGF), bone morphogenic protein (BMP), basic fibroblast growth factor (bFGF), insulin and 3-isobutyl-1-methylxasthine (IBMX). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R, Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag, and these are hereby incorporated by reference in their entirety.

In one embodiment, the composition can include one or more bioactive agents to induce healing or regeneration of damaged heart tissue, such as recruiting blood vessel forming cells from the surrounding tissues to provide connection points for the nascent vessels. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. Other bioactive agents can promote increased mitosis for cell growth and cell differentiation.

A great number of growth factors and differentiation factors are known in the art to stimulate cell growth and differentiation of stem cells and progenitor cells. Suitable growth factors and cytokines include any cytokines or growth factors capable of stimulating, maintaining, and/or mobilizing progenitor cells. They include but are not limited to stem cell factor (SCF), granulocyte-colony stimulating factor (G-CSF), granulocyte-macrophage stimulating factor (GM-CSF), stromal cell-derived factor-1, steel factor, vascular endothelial growth factor (VEGF), TGFβ, platelet derived growth factor (PDGF), angiopoietins (Ang), epidermal growth factor (EGF), bone morphogenic protein (BMP), fibroblast growth factor (FGF), hepatocyte growth factor (HGF), insulin-like growth factor (IGF-1), interleukin (IL)-3, IL-la, IL-13, IL-6, IL-7, IL-8, IL-11, and IL-13, colony-stimulating factors, thrombopoietin, erythropoietin, fit3-ligand, and tumor necrosis factor α. Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In one embodiment of all aspects of the therapeutic methods described, the composition described is a suspension of recombinant CPCs in a suitable physiologic carrier solution such as saline. The suspension can contain additional bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof.

In certain embodiments of all aspects of the therapeutic methods described, the bioactive agent is a “pro-angiogenic factor,” which refers to factors that directly or indirectly promote new blood vessel formation in the heart. The pro-angiogenic factors include, but are not limited to ephrins (e.g., ephrin A or ephrin B), epidermal growth factor (EGF), E-cadherin, VEGF, angiogenin, angiopoietin-1, fibroblast growth factors: acidic (aFGF) and basic (bFGF), fibrinogen, fibronectin, heparanase, hepatocyte growth factor (HGF), angiopoietin, hypoxia-inducible factor-1 (HIF-1), insulin-like growth factor-1 (IGF-1), IGF, BP-3, platelet-derived growth factor (PDGF), VEGF-A, VEGF-C, pigment epithelium-derived factor (PEDF), vascular permeability factor (VPF), vitronection, leptin, trefoil peptides (TFFs), CYR61 (CCN1), NOV (CCN3), leptin, midkine, placental growth factor platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), c-Myc, granulocyte colony-stimulating factor (G-CSF), stromal derived factor 1 (SDF-1), scatter factor (SF), osteopontin, stem cell factor (SCF), matrix metalloproteinases (MMPs), thrombospondin-1 (TSP-1), pleitrophin, proliferin, follistatin, placental growth factor (PIGF), midkine, platelet-derived growth factor-BB (PDGF), and fractalkine, and inflammatory cytokines and chemokines that are inducers of angiogenesis and increased vascularity, e.g., interleukin-3 (IL-3), interleukin-8 (IL-8), CCL2 (MCP-1), interleukin-8 (IL-8) and CCL5 (RANTES).

Suitable dosage of one or more therapeutic agents in the compositions described herein can include a concentration of about 0.1 to about 500 ng/ml, about 10 to about 500 ng/ml, about 20 to about 500 ng/ml, about 30 to about 500 ng/ml, about 50 to about 500 ng/ml, or about 80 ng/ml to about 500 ng/ml. In some embodiments, the suitable dosage of one or more therapeutic agents is about 10, about 25, about 45, about 60, about 75, about 100, about 125, about 150, about 175, about 200, about 225, about 250, about 275, about 300, about 325, about 350, about 375, about 400, about 425, about 450, about 475, or about 500 ng/ml. In other embodiments, suitable dosage of one or more therapeutic agents is about 0.6, about 0.7, about 0.8, about 0.9, about 1.0, about 1.5, or about 2.0 μg/ml.

In some embodiments of all aspects of the therapeutic methods described, the standard therapeutic agents for heart disease are those that have been described in detail, see, e.g., Harrison's Principles of Internal Medicine, 15th edition, 2001, E. Braunwald, et al., editors, McGraw-Hill, New York, N.Y., ISBN 0-07-007272-8, especially chapters 252-265 at pages 1456-1526; Physicians Desk Reference 54th edition, 2000, pages 303-3251, ISBN 1-56363-330-2, Medical Economics Co., Inc., Montvale, N.J. Treatment of any heart disease or disorder can be accomplished using the treatment regimens described herein. For chronic conditions, intermittent dosing can be used to reduce the frequency of treatment. Intermittent dosing protocols are as described herein.

For the clinical use of the methods described herein, isolated populations of recombinant CPCs described herein can be administered along with any pharmaceutically acceptable compound, material, carrier or composition which results in an effective treatment in the subject. Thus, a pharmaceutical formulation for use in the methods described herein can contain an isolated recombinant CPCs in combination with one or more pharmaceutically acceptable ingredients.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent, media (e.g., stem cell media), encapsulating material, manufacturing aid (e.g., lubricant, talc magnesium, calcium or zinc stearate, or steric acid), or solvent encapsulating material, involved in maintaining the activity of, carrying, or transporting the isolated recombinant CPCs from one organ, or portion of the body, to another organ, or portion of the body.

Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) phosphate buffered solutions; (3) pyrogen-free water; (4) isotonic saline; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) cellulose, and its derivatives, such as sodium carboxymethyl cellulose, methylcellulose, ethyl cellulose, microcrystalline cellulose and cellulose acetate; (17) powdered tragacanth; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C2-C12 alcohols, such as ethanol; (25) starches, such as corn starch and potato starch; and (26) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some aspects, the invention provides methods of producing recombinant CPCs comprising one, two or three copies of a tumor suppressor p53 gene in addition to the endogenous copy of a p53 gene.

In some embodiments, the invention provides a method of producing a large quantity of cardiac progenitor cells (CPCs) comprising: (a) isolating CPCs from heart tissue: (b) introducing one or more tumor suppressor p53 genes into the CPCs of step (a); and (c) culturing and expanding the CPCs of step (b), thereby producing a large quantity of CPCs.

In one embodiment, the invention provides a method of promoting cellular engraftment and growth of cells in an organ or tissue during cell therapy, comprising: (a) extracting cells from an organ or tissue; (b) introducing one or more tumor suppressor p53 genes into the cells of step (a); (c) culturing and expanding said cells from step (b); and (d) applying an amount of said cells from step (c) to an area of damaged organ or tissue, thereby promoting cellular engraftment and growth of cells in the damaged organ or tissue.

In one embodiment, the invention provides a method of producing isolated cardiac progenitor cells (CPCs) having an improved ability to tolerate oxidative stress, comprising: (a) isolating CPCs from heart tissue; (b) introducing one or more tumor suppressor p53 genes into the CPCs of step (a); and (c) culturing and expanding the CPCs of step (b), thereby producing CPCs having an improved ability to tolerate oxidative stress compared to CPCs from step (a).

In one embodiment, the invention provides a method of producing isolated cardiac progenitor cells (CPCs) having restored DNA integrity, comprising: (a) isolating CPCs from heart tissue; (b) introducing one or more tumor suppressor p53 genes into the CPCs of step (a); and (c) culturing and expanding the CPCs of step (b), thereby producing CPCs having restored DNA integrity compared to CPCs from step (a).

In one embodiment, the invention provides a method of producing isolated cardiac progenitor cells (CPCs) having an improved proliferative capacity, comprising: (a) isolating CPCs from heart tissue; (b) introducing one or more tumor suppressor p53 genes into the CPCs of step (a); and (c) culturing and expanding the CPCs of step (b), thereby producing CPCs having an improved proliferative capacity compared to CPCs from step (a).

In some embodiments, one or more exogenous tumor suppressor p53 genes may be introduced into CPCs isolated from a subject with heart disease to generate recombinant CPCs. These recombinant CPCs may then be administered to the subject from whom the parental CPCs were isolated to treat the subject's heart disease.

The one or more exogenous tumor suppressor p53 genes may be introduced into CPCs by any suitable methods of genetic engineering. For example, the p53 gene may be introduced via a viral vector, a plasmid or a nanoparticle. An exogenous p53 gene may be operatively linked to a constitutive promoter, an inducible promoter or a cardiac-tissue-specific promoter. In some embodiments, an exogenous p53 gene integrates into the genome of the recombinant CPC.

Unless otherwise defined, all technical and scientific terms used herein have the same meaning as commonly understood by one of ordinary skill in the art to which this invention belongs. Certain terms employed herein, in the specification, examples and claims are collected here.

As used herein, “in vivo” (Latin for “within the living”) refers to those methods using a whole, living organism, such as a human subject. As used herein, “ex vivo” (Latin: out of the living) refers to those methods that are performed outside the body of a subject, and refers to those procedures in which an organ, cells, or tissue are taken from a living subject for a procedure, e.g., isolating recombinant CPCs from heart tissue obtained from a donor subject, and then administering the isolated recombinant CPCs to a recipient subject. As used herein, “in vitro” refers to those methods performed outside of a subject, such as an in vitro cell culture experiment. For example, recombinant CPCs can be cultured in vitro to expand or increase the number of recombinant CPCs, or to direct differentiation of the CPCs to a specific lineage or cell type, e.g., cardiomyocytes, prior to being used or administered according to the methods described herein.

The term “pluripotent” as used herein refers to a cell with the capacity, under different conditions, to commit to one or more specific cell type lineage and differentiate to more than one differentiated cell type of the committed lineage, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells) also have the characteristic of the capacity of extended passaging without loss of growth potential, relative to primary cell parents, which generally have capacity for only a limited number of divisions in culture.

The term “progenitor” cell are used herein refers to cells that have a cellular phenotype that is more primitive (i.e., is at an earlier step along a developmental pathway or progression than is a fully differentiated or terminally differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. Progenitor cells give rise to precursor cells of specific determinate lineage, for example, certain cardiac progenitor cells divide to give cardiac cell lineage precursor cells. These precursor cells divide and give rise to many cells that terminally differentiate to, for example, cardiomyocytes.

The term “precursor” cell is used herein refers to a cell that has a cellular phenotype that is more primitive than a terminally differentiated cell but is less primitive than a stem cell or progenitor cell that is along its same developmental pathway. A “precursor” cell is typically progeny cells of a “progenitor” cell which are some of the daughters of “stem cells”. One of the daughters in a typical asymmetrical cell division assumes the role of the stem cell.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that the cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” is used to refer to any multipotent stem cell derived from non-embryonic tissue, including juvenile and adult tissue. In some embodiments, adult stem cells can be of non-fetal origin.

In the context of cell ontogeny, the adjective “differentiated” or “differentiating” is a relative term meaning a “differentiated cell” is a cell that has progressed further down the developmental pathway than the cell it is being compared with. Thus, stem cells can differentiate to lineage-restricted precursor cells (such as a cardiac stem cell), which in turn can differentiate into other types of precursor cells further down the pathway (such as an exocrine or endocrine precursor), and then to an end-stage differentiated cell, which plays a characteristic role in a certain tissue type, and may or may not retain the capacity to proliferate further. The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a CPC) in a cellular differentiation process.

As used herein, the term “somatic cell” refers to any cell forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

As used herein, the term “adult cell” refers to a cell found throughout the body after embryonic development.

The term “phenotype” refers to one or a number of total biological characteristics that define the cell or organism under a particular set of environmental conditions and factors, regardless of the actual genotype. For example, the expression of cell surface markers in a cell. The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The terms “renewal” or “self-renewal” or “proliferation” are used interchangeably herein, are used to refer to the ability of stem cells to renew themselves by dividing into the same non-specialized cell type over long periods, and/or many months to years.

In some instances, “proliferation” refers to the expansion of cells by the repeated division of single cells into two identical daughter cells.

The term “lineages” is used herein describes a cell with a common ancestry or cells with a common developmental fate.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “tissue” refers to a group or layer of specialized cells which together perform certain special functions. The term “tissue-specific” refers to a source of cells from a specific tissue.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” typically means a decrease by at least about 5%-10% as compared to a reference level, for example a decrease by at least about 20%, or at least about 30%, or at least about 40%, or at least about 50%, or at least about 60%, or at least about 70%, or at least about 80%, or at least about 90% decrease (i.e., absent level as compared to a reference sample), or any decrease between 10-90% as compared to a reference level. In the context of treatment or prevention, the reference level is a symptom level of a subject in the absence of administering a population of recombinant CPCs.

The terms “increased”, “increase” or “enhance” are all used herein to generally mean an increase by a statically significant amount; for the avoidance of any doubt, the terms “increased”, “increase” or “enhance” means an increase of at least 10% as compared to a reference level, for example an increase of at least about 20%, or at least about 30%, or at least about 40%, or at least about 50%, or at least about 60%, or at least about 70%, or at least about 80%, or at least about 90% increase or more, or any increase between 10-90% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of recombinant CPCs expansion in vitro, the reference level is the initial number of recombinant CPCs isolated from a heart sample or generated by genetic engineering.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present invention was performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R Ian Freshney, Publisher: Wiley-Liss; 5th edition (2005) and Animal Cell Culture Methods (Methods in Cell Biology, Vol. 57, Jennie P. Mather and David Barnes editors, Academic Press, 1st edition, 1998) which are all herein incorporated by reference in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.”

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously.

The invention will be further clarified by the following examples, which are intended to be purely exemplary and in no way limiting.

### EXAMPLES

### Example 1: Methods

### 1.1. Animals

All procedures were approved by the Institutional Animal Care and Use Committee of the Brigham and Women's Hospital. Animals received humane care in compliance with the “Guide for the Care and Use of Laboratory Animals” as described by the Institute of Laboratory Animal Research Resources, Commission on Life Sciences, National Research Council. Male and female wild-type (WT) and super p53 transgenic (p53-tg) mice in a C57BL/6 genetic background were studied (Garcia-Cao et al., 2002, Garcia-Cao et al., 2006). WT and p53-tg at different ages were included in the protocols.

### 1.2. Ventricular Hemodynamics

Cardiac function was measured in young-adult, 3-6 months of age, and old, 24-31 months of age, WT and p53-tg mice. Left ventricular (LV) parameters (Leri et al., 2003, Torella et al., 2004, Rota et al., 2007, Sanada et al., 2014) were obtained in the closed chest preparation with a MPVS-400 system for small animals (Millar Instruments) equipped with a PVR-1045 catheter. Under sodium pentobarbital (50 mg/kg body weight, i.p.) anesthesia, the right carotid artery was exposed and the pressure transducer was inserted in the carotid artery and advanced into the LV cavity. Data were acquired and analyzed with Chart 5 (ADInstruments) software

### 1.3 Myocyte Isolation

Under pentobarbital anesthesia, the heart was excised and LV myocytes were enzymatically dissociated (Torella et al., 2004, Rota et al., 2007, Signore et al., 2015). Briefly, the myocardium was perfused retrogradely through the aorta at 37° C. with a Ca²⁺-free solution gassed with 85% Oz and 15% N₂. After 5 min, 0.1 mM CaCl₂, 274 units/ml collagenase (type 2, Worthington Biochemical Corp.) and 0.57 units/ml protease (type XIV, Sigma) were added to the solution which contained (mM): NaCl 126, KCl 4.4, MgCl₂ 5, HEPES 20, Glucose 22, Taurine 20, Creatine 5, Na Pyruvate 5 and NaH₂PO₄ 5 (pH 7.4, adjusted with NaOH). At completion of digestion, the LV was cut in small pieces and re-suspended in Ca²⁺ 0.1 mM solution. Myocytes were collected by differential centrifugation.

1.4 Ca²⁺ Transients and Sarcomere Shortening Isolated LV myocytes were placed in a bath on the stage of an Axiovert Zeiss Microscope and IX71 Olympus inverted microscope for the measurements of contractility and Ca²⁺ transients. Experiments were conducted at room temperature. Cells were bathed continuously with a Tyrode solution containing (mM): NaCl 140, KCl 5.4, MgCl₂ 1, HEPES 5, Glucose 5.5 and CaCl₂) 1.0 (pH 7.4, adjusted with NaOH). Measurements were performed in field-stimulated cells by using IonOptix fluorescence and contractility systems (IonOptix, Milton, Mass.). Contractions were elicited by rectangular depolarizing pulses, 2 ms in duration, and twice-diastolic threshold in intensity, by platinum electrodes (Torella et al., 2004, Signore et al., 2015). Changes in mean sarcomere length were computed by determining the mean frequency of sarcomere spacing by fast Fourier transform and then frequency data were converted to length. Ca²⁺ transients were measured by epifluorescence after loading the myocytes with 10 μM Fluo-3 AM (Invitrogen). Excitation length was 480 nm with emission collected at 535 nm using a 40× oil objective. Fluo-3 signals were expressed as normalized fluorescence (F/F₀).

### 1.5 Immunohistochemistry

Following the acquisition of the hemodynamic parameters, the abdominal aorta was cannulated with a polyethylene catheter, PE-50, filled with a phosphate buffer, 0.2 M, pH 7.4, and heparin, 100 U/ml. In rapid succession, the heart was arrested in diastole by the injection of 0.15 ml of CdCl₂, 100 mM, through the aortic catheter, the thorax was opened, perfusion with phosphate buffer was started, and the vena cava was cut to allow drainage of blood and perfusate. After perfusion with phosphate buffer for 2 min, the coronary vasculature was perfused for 15 min with formalin. Subsequently, the heart was excised and embedded in paraffin (Leri et al., 2003, Torella et al., 2004, Rota et al., 2007, Sanada et al., 2014).

Formalin-fixed paraffin-embedded myocardial sections were labeled with goat polyclonal anti-c-kit (R&D: cat. no. AF1356), mouse monoclonal anti-α-sarcomeric actin (Sigma-Aldrich: clone 5C5, cat. no. A2172) to identify CPCs and cardiomyocytes, respectively. Nuclei were stained by DAPI. Cycling CPCs and cardiomyocytes were recognized by labeling with mouse monoclonal anti-Ki67 antibody (BD Biosciences: cat. no. 550609). Apoptotic and senescent cells were recognized by the TUNEL assay (Roche: cat. no. 11684795910) and p16^{INK4a} localization (Cell Signaling: cat. no. 4824), respectively (Leri et al., 2003, Torella et al., 2004, Rota et al., 2007, Sanada et al., 2014). The number of c-kit-positive CPCs per unit area of myocardium in the atria and LV mid-region was determined as previously described (Torella et al., 2004, Sanada et al., 2014).

### 1.6 Western Blotting of Cardiomyocytes

Protein lysates of cardiomyocytes were obtained using RIPA buffer (Sigma) and protease inhibitors. Equivalents of 50 gig of proteins were separated on 10-12% SDS-PAGE, transferred onto PVDF membranes (Bio-Rad) and subjected to Western blotting with mouse monoclonal anti-Aogen (Swant: cat. no. 138), rabbit polyclonal anti-ATIR (Millipore: cat. no. 15552), rabbit polyclonal anti-Bax (Cell Signaling: cat. no. 7074) and rabbit polyclonal anti-Bcl2 (Cell Signaling: cat. no. D17C4) diluted 1:500-1000 in TBST or BSA overnight at 4° C. HRP-conjugated anti-IgG were used as secondary antibodies. Proteins were detected by chemiluminescence (SuperSignal West Femto Maximum Sensitivity Substrate, Thermo Scientific: cat. no. 34095) and optical density was measured. Loading conditions were determined by Ponceau S (Sigma) staining of the membrane after transfer. Lung and kidney were used as positive controls for Aogen and ATIR, respectively. SVT2 and B16 melanoma cells were employed for the recognition of the bands corresponding to Bax and Bcl2, respectively (Leri et al., 1998, Torella et al., 2004, Goichberg et al., 2013).

### 1.7 CPC Isolation and Expansion

Following myocyte isolation, the small cardiac cell pool present in the supernatant was plated in Petri dishes and, 24 h later, c-kit positive cells were obtained by immunomagnetic sorting (Miltenyi Biotec.: cat. no. 130-091-224) (Beltrami et al., 2003, D'Amario et al., 2011, D'Amario et al., 2014, Sanada et al., 2014). Subsequently, c-kit-positive cells were cultured in F12K medium supplemented with 10% fetal bovine serum. Immunomagnetic sorting for c-kit was repeated every three passages to select with this protocol the fraction of cells that retained c-kit expression. This approach was required because mouse c-kit-positive CPCs tend to lose this surface receptor with time in culture. When possible, immediately sorted cells were utilized; however, assays requiring large numbers of CPCs were conducted after cell expansion.

### 1.8 Population Doubling Time (PDT)

CPCs were plated at low density. The number of cells per unit area was determined at the time of seeding and 24 h later (D'Amario et al., 2011, D'Amario et al., 2014). PDT was computed by linear regression of log 2 values of cell number.

### 1.9 Proliferation, Senescence and Apoptosis

These cellular parameters were measured in baseline conditions, following exposure to doxorubicin (Doxo; 0.5 μM) for 4 h, and 24, 48 and 72 h following removal of Doxo. Cells were fixed in 4% paraformaldehyde and the fraction of cycling cells was determined by immunolabeling for Ki67 (eBioscience: cat. no. 14-5698-82, RRID: AB_10854564) and confocal microscopy (D'Amario et al., 2011, D'Amario et al., 2014, Goichberg et al., 2013). The fraction of cells that reached replicative senescence and irreversible growth arrest was evaluated by the expression of the senescence-associated protein p16^{INK4a} (Abcam: cat no. ab16123, RRID: AB_302274) (D'Amario et al., 2011, D'Amario et al., 2014, Goichberg et al., 2013). Apoptosis was measured in CPCs at baseline and following exposure to Doxo with the Annexin V detection assay (BD Pharmingen). Annexin V binds to the phosphatidylserine exposed on the outer leaflet of the cell membrane during apoptotic cell death. CPCs were seeded in 96 multi-well clear bottom black plates (3603, Corning); 24 h later, the medium was removed and cells were washed with PBS. FITC-Annexin V (556547, Pharmingen) diluted in binding buffer provided by the manufacturer was then added to the wells for a period of 30 min. After washing in PBS, cells were stained with DAPI. FITC (Excitation 490 nm; Emission 525 nm) and DAPI (Excitation 358 nm; Emission 461 nm) signals were quantified using Perkin Elmer EnVision Multilabel Reader. Apoptosis was calculated by normalizing the FITC signal to the number of cells represented by the DAPI signal.

### 1.10 DDR Foci and Comet Assay

CPCs were stained with a mouse anti-phospho-histone H2A.X (Ser139) (Millipore: cat. no. 05-636, RRID: AB_309864). Imaris software spot module was employed for the recognition of the γH2A.X-positive DDR foci and 3D rendering of the data (Goichberg et al., 2013). The number of foci per nucleus was counted utilizing the Imaris software.

The comet assay was performed utilizing the OxiSelect Comet Assay Kit (Cell Biolabs: cat. no. STA-351). Cells were embedded in agarose gel and placed on top of a microscope slide. Slides were treated with alkaline lysis buffer to remove proteins and, subsequently, immersed in TE buffer. Electrophoresis was performed to induce the formation of comets. Slides were stained with Vista green dye and analyzed by fluorescence microscopy (Lorenzo et al., 2013). The distance between the center of the head and the center of the tail, i.e. the tail moment length, was measured with ImageJ using comet assay plug-in. The tail moment was then calculated by the product of the percentage of damaged DNA and the tail moment length.

### 1.11 Western Blotting of CPCs

Protein lysates of CPCs were obtained using RIPA buffer (Sigma-Aldrich: cat. no. R0278) and protease inhibitors (Torella et al., 2004, Goichberg et al., 2013). Equivalents of 10 μg proteins were separated on 4-20% SDS-PAGE and subjected to traditional Western blotting. Additionally, equivalents of 1 gig proteins were analyzed with ProteinSimple Wes automated Western blotting system (Harris, 2015). The following antibodies were utilized: mouse monoclonal anti-p53 (Cell Signaling), rabbit polyclonal anti-p53 (Ser 37) (Cell Signaling Technology: cat. no. 2524, RRID: AB_331743), rabbit polyclonal anti-p53 (Ser15) (Cell Signaling Technology: cat. no. 9286S, RRID: AB_331741) and mouse monoclonal anti-p16^{INK4a} (Cell Signaling Technology: cat. no. 8884S, RRID: AB_11129865). Loading conditions were determined by GAPDH

1.12 qRT-PCR

Total RNA was extracted from CPCs with TRIzol Reagent (Invitrogen: cat. no. 15596018) and employed for the measurement of the quantity of transcripts of p53, Mdm2, Puma, Noxa, PIDD, Trp53inp, p16^{INK4a}, p21^{Cip1}, IGF-1 and PCNA. cDNA for mRNAs was obtained from 2 μg total RNA in a 20 μl reaction using High Capacity cDNA Reverse Transcription Kit (Applied Biosystems: cat. no. 4368814) and 100 pmole of oligo(dT)₁₅ primer (Hosoda et al., 2009, Goichberg et al., 2013). This mixture was incubated at 37° C. for 2 h. Quantitative RT-PCR was performed with primers designed using the Vector NTI (Invitrogen) software or downloaded from the NIH qdepot mouse primer database (for sequences see Supplementary Methods). StepOnePlus Real-Time PCR system (Applied Biosystems) was employed. cDNA synthesized from 100 ng total RNA was combined with Power SYBR Green PCR Master Mix (Applied Biosystems: cat. no. 4367659) and 0.5 μM each of forward and reverse primers. Cycling conditions were as follows: 95° C. for 10 min followed by 40 cycles of amplification (95° C. denaturation for 15 s, and 60° C. annealing-extension for 1 min). The melting curve was then obtained. To avoid the influence of genomic contamination, forward and reverse primers for each gene were located in different exons. Reactions with primers alone were also included as negative controls. Quantified values were normalized against the input determined by the housekeeping gene 12-microglobulin. Real-time PCR products were run on 2% agarose/IX TBE gel.

qRT-PCR Primer Sequences

Mouse p16INK4a
F:
(SEQ ID NO: 1)
5′-CGTGAACATGTTGTTGAGGC-3′
R:
(SEQ ID NO: 2)
5′-GCAGAAGAGCTGCTACGTGA-3′
Mouse Igf1
F:
(SEQ ID NO: 3)
5′-TGGATGCTCTTCAGTTCGTG-3′
R:
(SEQ ID NO: 4)
5′-CACTCATCCACAATGCCTGT-3′
Mouse H2A X
F:
(SEQ ID NO: 5)
5′-GGTCAGAGAGACGCTTACCG-3′
R:
(SEQ ID NO: 6)
5′-GTAGTTGAGTCGCTGGGGAA-3′
Mouse p21
F:
(SEQ ID NO: 7)
5′-CCAGGATTGGACATGGTGCC-3′
R:
(SEQ ID NO: 8)
5′-GTGAGGAGGAGCATGAATGGAG-3′
Mouse Puma
F:
(SEQ ID NO: 9)
5′-CGGGCTAGACCCTCTACG-3′
R:
(SEQ ID NO: 10)
5′-AGCCCTCCAGAAGGCAAC-3′
Mouse Noxa
F:
(SEQ ID NO: 11)
5′-TTCAAGTCTGCTGGCACCCG-3′
R:
(SEQ ID NO: 12)
5′-AACGCGCCAGTGAACCCAAC-3′
Mouse p53
F:
(SEQ ID NO: 13)
5′-CTAGCATTCAGGCCCTCATC-3′
R:
(SEQ ID NO: 14)
5′-TCCGACTGTGACTCCTCCAT-3′
Mouse PCNA
F:
(SEQ ID NO: 15)
5′-TGGATAAAGAAGAGGAGGCG-3′
R:
(SEQ ID NO: 16)
5′-GGAGACAGTGGAGTGGCTTT-3′
Mouse PIDD
F:
(SEQ ID NO: 17)
5′-AAGGTTCCGTGGAGTCTGCT-3′
R:
(SEQ ID NO: 18)
5′-CAGAGTGGTCAGGGTTCCAT-3′
Mouse Trp53inp1
F:
(SEQ ID NO: 19)
5′-CTACCTCAGCACCCGCAG-3′
R:
(SEQ ID NO: 20)
5′-GCCCAATATCACAGACGAGA-3′
Mouse Mdm2
F:
(SEQ ID NO: 21)
5′-TCTGTGAAGGAGCACAGGAA-3′
R:
(SEQ ID NO: 22)
5′-CTGCTCTCACTCAGCGATGT-3′
Mouse b2-M
F:
(SEQ ID NO: 23)
5′-ATGTGAGGCGGGTGGAACG-3′
R:
(SEQ ID NO: 24)
5′-CTCGGTGACCCTGGTCTTTTG-3′

### 1.13 Diabetes and CPC Injection

C57Bl/6 female mice at 3-4 months of age were treated with streptozotocin (STZ, Sigma) for 7 consecutive days (˜100 mg/kg body weight per day, i.p.) (Rota et al., 2006). STZ was dissolved in 0.9/o saline solution containing 20 mM/1 sodium citrate tribasic dehydrate (Sigma). Final STZ concentration was 5 mg/I. Animals developed hyperglycemia ˜2 weeks after the last injection of STZ. TRUEtrack meter (Home Diagnostics, Inc.) and test strips were employed to measure blood glucose. Animals with blood glucose level >400 mg/dl were included in the study.

Three-four weeks after the onset of hyperglycemia, 200,000 CPCs were injected within the myocardium (4 injections of 5 μl each). Mice were sacrificed 3 days following cell transplantation. Hearts were perfused with formalin and embedded in paraffin as described above. Tissue sections obtained from the mid-portion of the LV were stained for GFP (rabbit polyclonal anti-GFP, Molecular Probes: cat. no. A-11122, RRID: AB_221569; chicken polyclonal anti-GFP, Abcam: ab13970, RRID: AB_300798), α-sarcomeric actin (mouse anti-α-sarcomeric actin IgM, Sigma-Aldrich: cat. no. A2172, RRID: AB_476695), GATA4 (rabbit polyclonal anti-GATA4, Abcam: cat. no. ab84593, RRID: AB_10670538) and troponin I (mouse monoclonal anti-troponin I, Abcam: cat. no. ab10231, RRID: AB_296967). The number of GFP-positive cells per 10 mm² of myocardium was measured throughout the entire cross-section of the LV.

### 1.14 Data Analysis

Data are presented as mean±SD. The Shapiro-Wilk test was utilized to define the normality of value distribution. In case of normal distribution, significance between two groups was determined by unpaired two-tailed Student's t-test. For multiple comparisons, the ANOVA test with the Bonferroni parametric correction was employed. When the normality test failed, the Mann-Whitney Rank Sum Test and the Kruskal-Wallis One Way ANOVA were employed. In all cases, p<0.05 was considered significant (McDonald, 2014).

### Example 2: Results

2.1 p53 does not Alter the Mechanical and Growth Properties of Cardiomyocytes

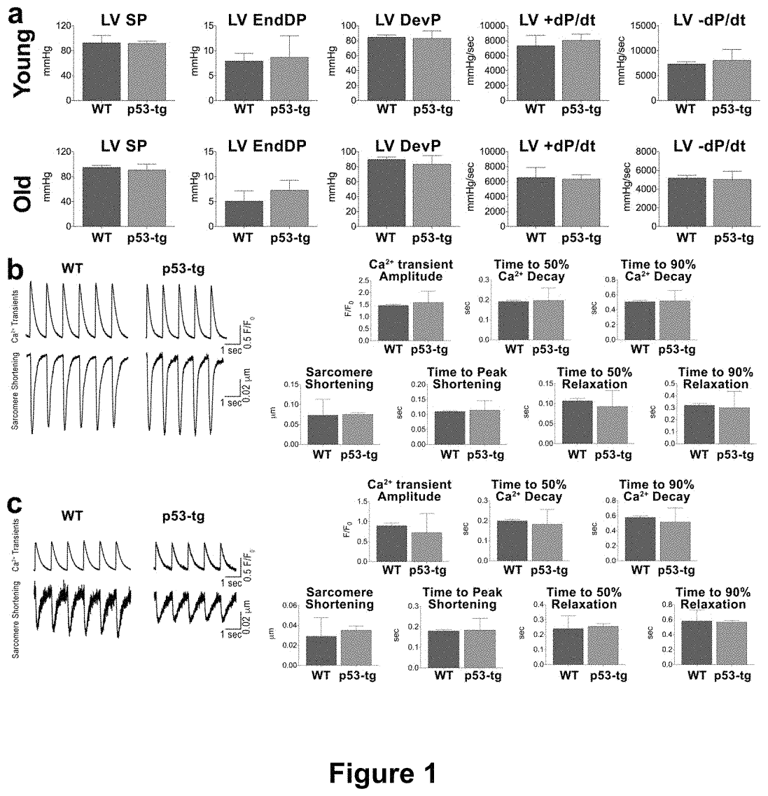

The overexpression of p53 results in premature organism aging and animal mortality (Serrano and Blasco, 2007). The shorter lifespan may be due to defects in cardiac performance and myocyte mechanics, commonly found in the old failing heart (Leri et al., 2003, Torella et al., 2004, Signore et al., 2015). Therefore, we determined whether an increase in p53 gene dosage had a negative effect on ventricular hemodynamics and the electro-mechanical properties of cardiomyocytes. Wild-type (WT) and p53-tg mice at 3-6 and 24-31 months of age were studied. At both ages, left ventricular (LV) systolic pressure, LV end-diastolic pressure, LV developed pressure, and LV+dP/dt and −dP/dt did not differ in p53-tg and WT mice (FIG. 1A).

Moreover, Ca²⁺ transient amplitude, sarcomere shortening, and the timing parameters of Ca²⁺ transient and sarcomere shortening were measured in isolated LV myocytes. In all cases, no differences were found (FIGS. 1B-1C), suggesting that the physiological properties of the LV and cardiomyocytes were preserved in WT mice as a function of age, and a single extra gene-dose of p53 did not alter the function of the old heart. These observations are consistent with previous results in which aging effects have not been detected in WT 26 month-old C57BL/6J mice (Sanada et al., 2014).

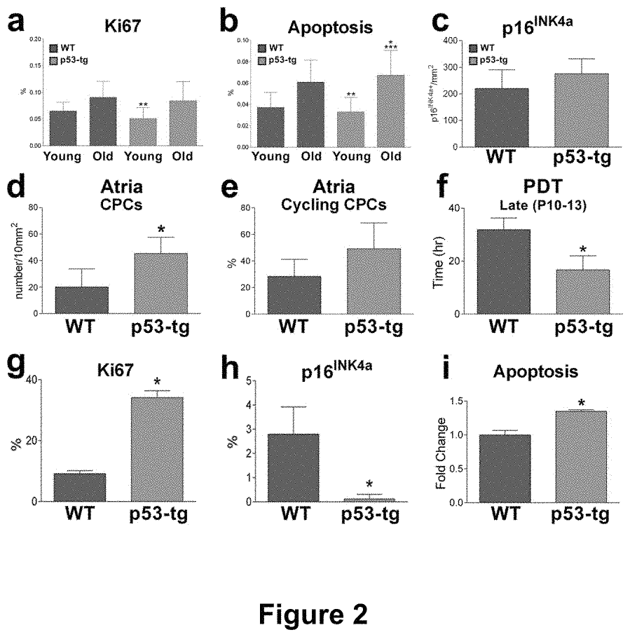

To define further the characteristics of cardiomyocytes, the degree of cell replication and death was evaluated in young-adult, 8-11 months, and old, 20-25 months, WT and p53-tg mice. The fraction of cycling Ki67-positive myocytes and the percentage of apoptotic myocytes were similar in young WT and p53-tg and increased equally with age in both groups of mice (FIG. 2A-2B). However, only the increase in cell death in p53-tg hearts was statistically significant. Moreover, the number of senescent p16^{INK4a}-positive cardiomyocytes was comparable in 18-33 month-old WT and p53-tg (FIG. 2C), supporting the notion that the extra copy of p53 did not promote myocardial aging. This finding is typical of this model in which the p53 transgene is physiologically regulated and it is not constitutively active. Conversely, transgenic and mutant mice with chronically active p53 signaling are characterized by shortened lifespan (Matheu et al., 2008).

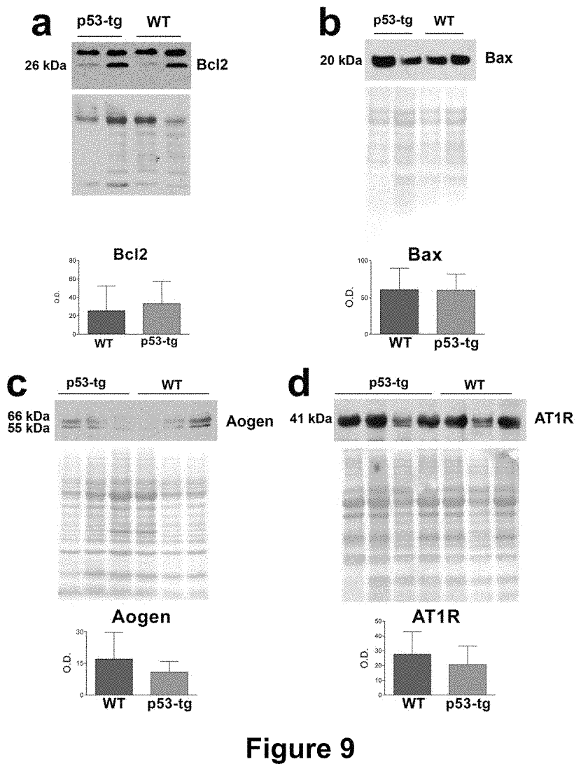

Cardiomyocyte apoptosis and aging are controlled in part by the expression of the p53-dependent genes, Bax and Bcl2, and the p53-regulated genes, angiotensinogen (Aogen) and angiotensin II (Ang II) type-1 receptors (AT1R) (Leri et al., 1998, Leri et al., 1999, Dimmeler and Leri, 2008, Xu et al., 2010). These parameters were measured in myocytes isolated from p53-tg and WT mice at 25 months of age. At the protein level, the quantity of the pro-apoptotic gene Bax and the anti-apoptotic gene Bcl2 was similar in WT and p53-tg myocytes (FIG. 9). Additionally, the levels of Aogen and ATIR did not differ in the two groups of cardiomyocytes (FIG. 9). Thus, an extra copy of p53 has no negative effects on cardiac performance, myocyte mechanics, Ca²⁺ transient, and cardiomyocyte growth, senescence and death.

### 2.2 p53 Preserves a Younger CPC Phenotype

CPC niches are preferentially located in the atrial myocardium (Sanada et al., 2014) so that a quantitative analysis was performed in this anatomical region of WT at 24-25 months and p53-tg at 24-31 months. The frequency of CPCs was significantly higher in p53-tg, while the fraction of replicating Ki67-positive CPCs was similar in the two groups (FIG. 2d, e). Because of these two variables, a larger pool of cycling CPCs was present in the atria of p53-tg mice.

To evaluate the growth reserve of CPCs, these cells were isolated from the myocardium of p53-tg at 26-30 months and WT at 23-25 months; cells were expanded in vitro and population doubling time (PDT) was determined at P10-P13. PDT was 47% shorter in p53-tg-CPCs than in WT-CPCs (FIG. 2f). Moreover, the percentage of Ki67-positive CPCs at P10-P13 was 3.9-fold higher in p53-tg-CPCs (1528/4561; 33.5%) than in WT-CPCs (543/6235; 8.7%) (FIG. 2g). At later passages, P16-P17, p16^{INK4a} comprised 2.9% of WT-CPCs (36/1255; 2.9%) and only 0.03% of p53-tg-CPCs (1/3275; 0.03%) (FIG. 2h). However, apoptosis was 35% higher in p53-tg-CPCs (FIG. 2i), despite the lower number of senescent cells. Thus, an extra copy of the p53 allele preserves a younger CPC phenotype after propagation in vitro and prevents the accumulation of senescent CPCs by potentiating cell death.

### 2.3 p53 Increases the Repair of DNA Damage in CPCs

Reactive oxygen species (ROS) induce foci of injury in the telomeric and non-telomeric DNA; this affects the growth and viability of the target cells (Schieber and Chandel, 2014). To evaluate whether p53-tg-CPCs had a superior, equal or inferior ability to sustain ROS-mediated DNA damage than WT-CPCs, these stem cell classes were exposed to a low dose of doxorubicin (Doxo) which is coupled with the formation of DNA strand breaks (Goichberg et al., 2013).

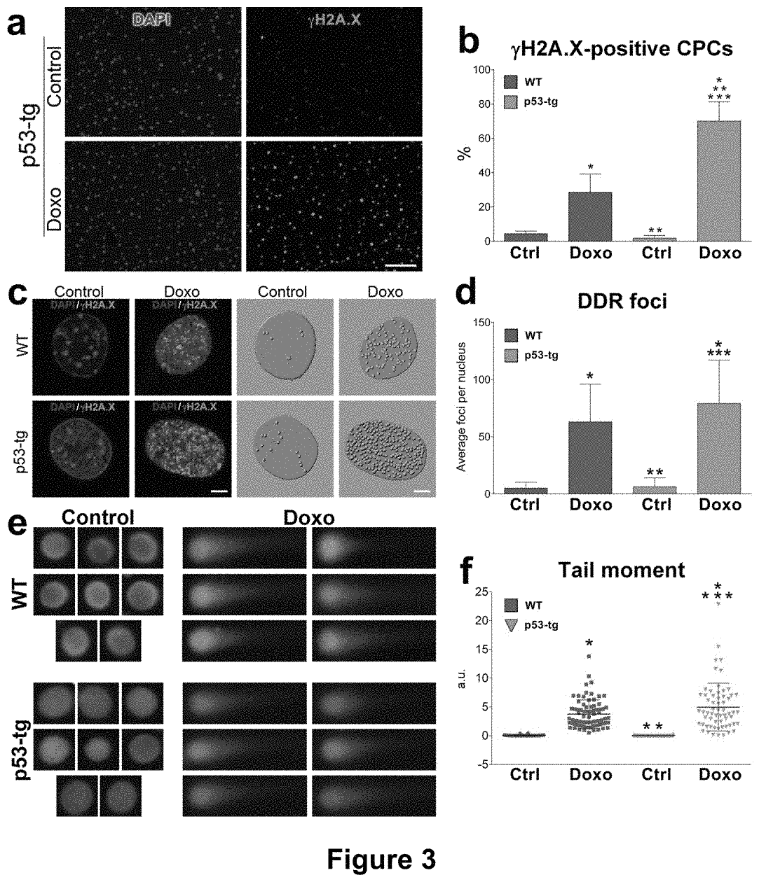

The γH2A.X protein accumulates at regions of DNA strand breaks, allowing the recognition of DNA damage (Mohrin et al., 2010, Goichberg et al., 2013). The localization of γH2A.X increased from 4.7% (200/4284; 4.7%) to 29% (1148/3958; 29%) in WT-CPCs and from 2.2% (296/13334; 2.2%) to 73.8% (12,185/16496; 73.8%) in p53-tg-CPCs (FIGS. 3A-3B). These results suggest that p53-tg-CPCs were 2.5-fold more efficient than WT-CPCs in recruiting γH2A.X at the sites of DNA damage, a process necessary for the initiation of DNA repair (Fumagalli et al., 2012). However, the enhanced recruitment of γH2A.X at the sites of DNA damage in p53-tg-CPCs may be independent from the extra copy of the p53 allele; p53-tg-CPCs possess a younger cell phenotype (see FIGS. 2H-2I), which may determine the higher efficiency of DNA repair in this progenitor cell class.

DDR foci correspond to the localization of the γH2A.X protein at the level of DNA lesions. In the presence of Doxo, the incidence of DDR foci per nucleus (p53-tgCPCs, baseline: 6.3; WT-CPCs, baseline: 5.1; p53-tgCPCs, Doxo: 79; WT-CPCs, Doxo: 63) increased markedly and in a similar manner, 12-fold, in p53-tg-CPCs and WT-CPCs (FIGS. 3C-3D), although a larger fraction of p53-tg-CPCs recruited γH2A.X, as shown in FIG. 3B. High values of DDR foci per nucleus may indicate an effective completion of DNA repair and/or a more extensive DNA damage (Lukas et al., 2011). To test this possibility the degree of DNA damage in the two categories of CPCs was determined by the Comet assay (Lorenzo et al., 2013).

CPCs were embedded in agarose on microscope slides and lysed to form nucleoids. Electrophoresis was performed to identify structures resembling comets at fluorescence microscopy (FIG. 3E). The fluorescence intensity of the tail (damaged DNA) relative to the head (intact DNA) reflects the percentage of DNA damage; 61-76 comets were analyzed in WT-CPCs and p53-tg-CPCs in the absence and presence of Doxo. The distance between the center of the head and the center of the tail, i.e. the tail moment length, indicates the frequency of DNA strand breaks. The tail moment was calculated by the product of the percentage of damaged DNA and the tail moment length. The tail moment provides a parameter that comprises both the extent of DNA damage and the frequency of DNA strand breaks; this index was found to be comparable at baseline and to increase similarly in p53-tg-CPCs and WT-CPCs following Doxo (FIG. 3F). Thus, the extent of damaged DNA promoted by oxidative stress was analogous in the two CPC classes (see FIG. 3D), but a larger fraction of cells carrying an extra copy of the p53 allele recruited γH2A.X (see FIG. 3B), possibly enhancing DNA repair.

### 2.4 p53 Enhances the Expression of Genes Regulating DDR

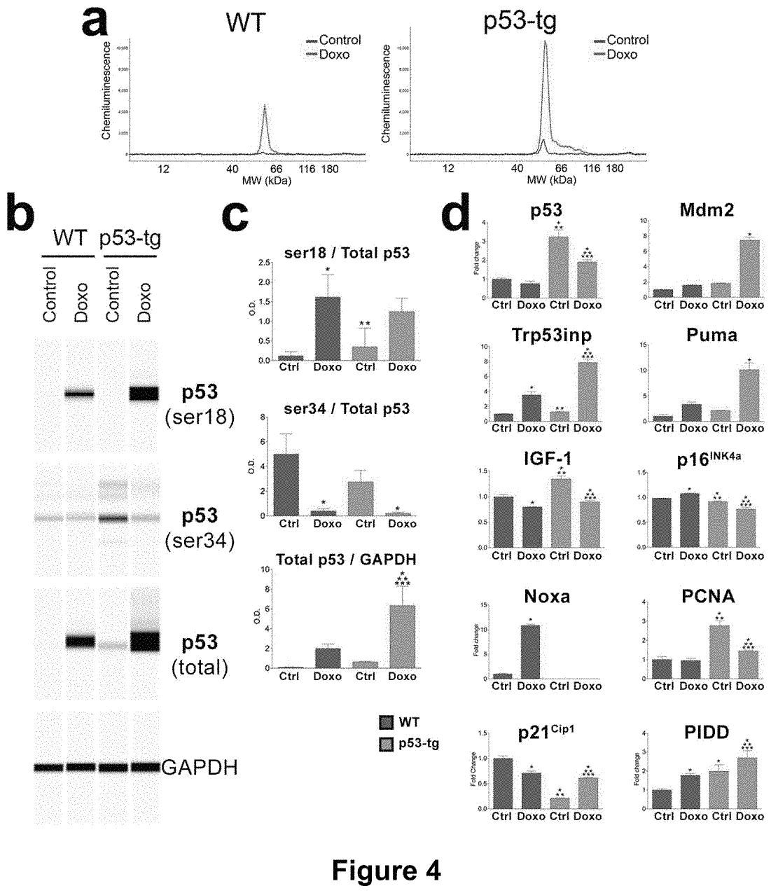

The tumor suppressor p53 trans-activates several genes implicated in the cell cycle and apoptosis (Riley et al., 2008), and an increase in p53 gene dosage may impact on the function of CPCs. Therefore, the expression of p53 and its target genes was measured in p53-tg-CPCs and WT-CPCs in the absence and presence of Doxo. At baseline, the quantity of p53 was similar in the two stem cell categories (FIGS. 4A-4C). After 4 h of Doxo, p53 levels increased and p53 phosphorylation at Ser-18, a post-translational modification required for p53 DNA binding, was present in both WT-CPCs and p53-tg-CPCs. At baseline, p53 phosphorylation at Ser-34 was high in WT-CPCs and in p53-tg-CPCs and with Doxo decreased in both stem cell categories (FIGS. 4B-4C). Together with other sites of post-translational modifications, phosphorylation of p53 at Ser-34 is relevant to DDR (Loughery and Meek, 2013).

The expression of p53 and other genes (Riley et al., 2008) implicated in inhibition of p53 activity (Mdm2), induction of apoptosis (Puma and Noxa), protection from oxidative stress (Trp53inp), cellular senescence (p16^{INK4a}), cell cycle arrest and DNA repair (p21^{Cip1}), and proliferation (IGF-1 and PCNA), was measured by qRT-PCR. The expression of PIDD was also determined; PIDD is a master regulator of cell fate decision, playing a critical role in DNA repair, cell proliferation, survival and death (Bock et al., 2012).

At baseline, p53, PIDD, IGF-1 and PCNA transcripts were higher and p21^{Cip1} was lower in p53-tg-CPCs than in WT-CPCs, possibly reflecting the enhanced proliferative activity of cells with an extra copy of the p53 gene (FIG. 4D). With Doxo treatment, Mdm2, Puma and p21^{Cip1} increased mostly in p53-tg-CPCs, suggesting that p21^{Cip1} promoted cell cycle arrest and favored DNA repair. However, p16^{INK4a} was decreased in p53-tg-CPCs. PIDD and Trp53inp were upregulated in p53-tg-CPCs and WT-CPCs, but the changes in Trp53inp were greater in p53-tg-CPCs; thus, the protection from oxidative stress was enhanced in p53-tg-CPCs (FIG. 4D). With Doxo, the expression of IGF-1 and PCNA decreased in p53-tg-CPCs and these changes are consistent with activation of the DNA repair process. In WT-CPCs, Doxo led to an attenuation of IGF-1 and an upregulation of Noxa, which may mediate cell apoptosis.

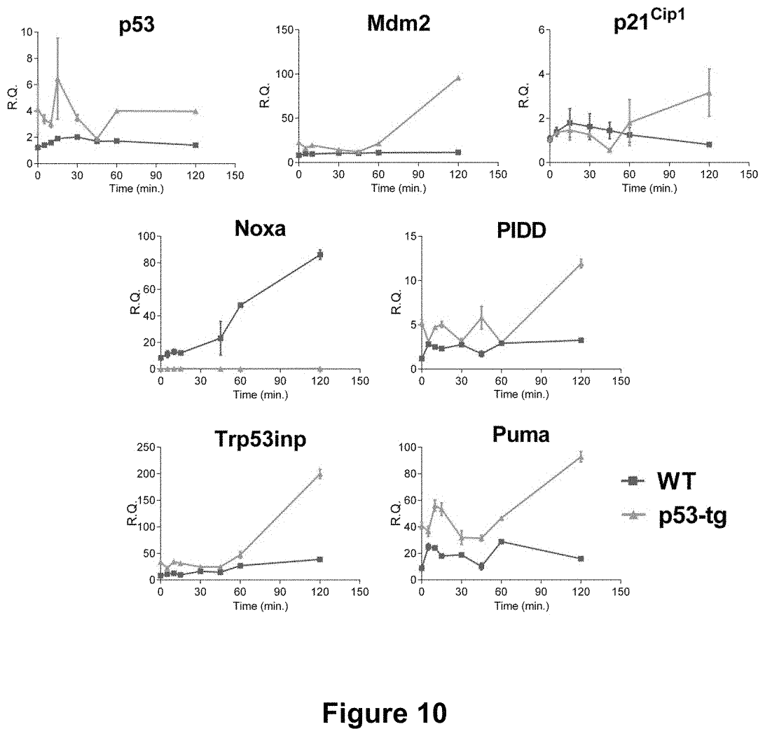

The temporal changes in the expression of p53, Mdm2, p21^{Cip1}, Noxa, PIDD, Trp53inp and Puma were evaluated in p53-tg-CPCs and WT-CPCs from time 0 to 120 min following Doxo-treatment (FIG. 10). In p53-tg-CPCs, the expression of p53 appeared to increase earlier than the upregulation of Mdm2, p21^{Cip1}, PIDD, Trp53inp and Puma. These adaptations suggest that oxidative stress was coupled with a rapid response in the genes modulating p53 function, growth arrest, oxidative DNA damage and repair, and cell death. Conversely, in WT-CPCs, the modest increase in p53 was associated with a time-dependent increase in the pro-apoptotic gene Noxa (FIG. 10).

The expression of Noxa and Puma is essential for p53-mediated apoptosis; in this regard, the deletion of these two genes prevents cell death in response to stimuli leading to upregulation of p53 activity (Valente et al., 2013). The differential expression of Noxa and Puma in WT-CPCs and p53-tg-CPCs with oxidative stress may depend on the distinct post-translational modifications of p53, which condition the transactivation of specific target genes. Additionally, γH2A.X, which is more effectively recruited at the sites of DNA damage in p53-tg-CPCs, promotes upregulation of Puma independently from p53 signaling (Xu et al., 2016). Thus, p53 is a critical determinant of stem cell fate and an extra copy of the p53 allele positively impacts on the survival and growth of CPCs.

### 2.5 p53 Promotes DNA Repair and Recovery of CPC Growth

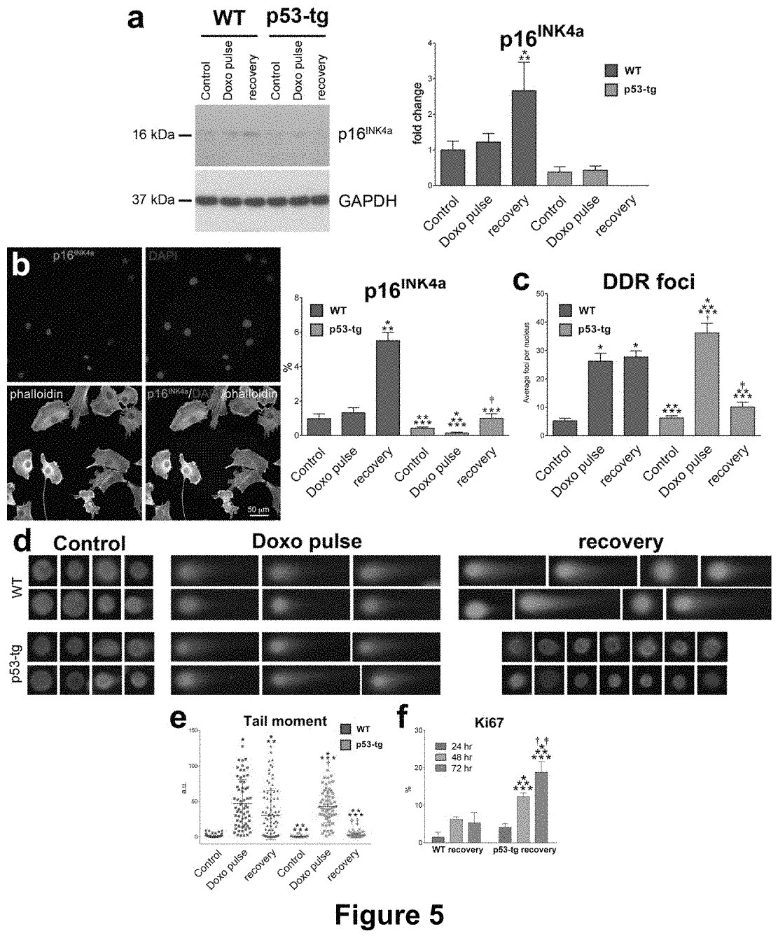

To determine whether the distinct response of CPC classes to oxidative stress was translated in a differential recovery in function, p53-tg-CPCs and WT-CPCs were exposed to Doxo for 4 h (Doxo-pulse) and, after Doxo removal, cellular senescence, DNA repair and proliferation were measured following a 72-hour recovery period (Recovery). After recovery, p16^{INK4a} expression was barely detectable by Western blotting in p53-tg-CPCs, but was upregulated in WT-CPCs (FIG. 5A). Similarly, by immunolabeling and confocal microscopy, a small fraction of p16^{INK4a}-positive cells was identified in p53-tg-CPCs, while numerous WT-CPCs expressed p16^{INK4a} [FIG. 5b; (WT-CPCs: control=6/610, 0.98%; Doxo pulse=22/1742, 1.26%; Recovery=150/2877, 5.2%) (p53-tg-CPCs: control=6/1903, 0.3.2%; Doxo pulse=3/4293, 0.07%; Recovery=32/3473, 0.9%)]. Importantly, following recovery, the number of DDR foci and the tail moment decreased dramatically in p53-tg-CPCs; however, these parameters remained high in WT-CPCs (FIGS. 5C-5E). Additionally, a progressive increase in cell proliferation was observed in p53-tg-CPCs from 24 to 48 and 72 h after the removal of Doxo [FIG. 5F; (WT-CPCs: 24 h=143/8167, 1.7%; 48 h=511/8405, 6.1%; 72 h=270/4915, 5.4%) (p53-tg-CPCs: 24 h=305/7902, 0.3.9%; 48 h=1443/13635, 11%; 72 h=1032/6246, 17%)]. In contrast, the reinstitution of cell proliferation was modest in WT-CPCs. Thus, following oxidative stress, an extra copy of the p53 allele potentiates the ability of CPCs to reestablish the integrity of the DNA, leading to a relevant restoration of cell growth.

### 2.6 p53 Increases the Engraftment of CPCs in the Diabetic Heart

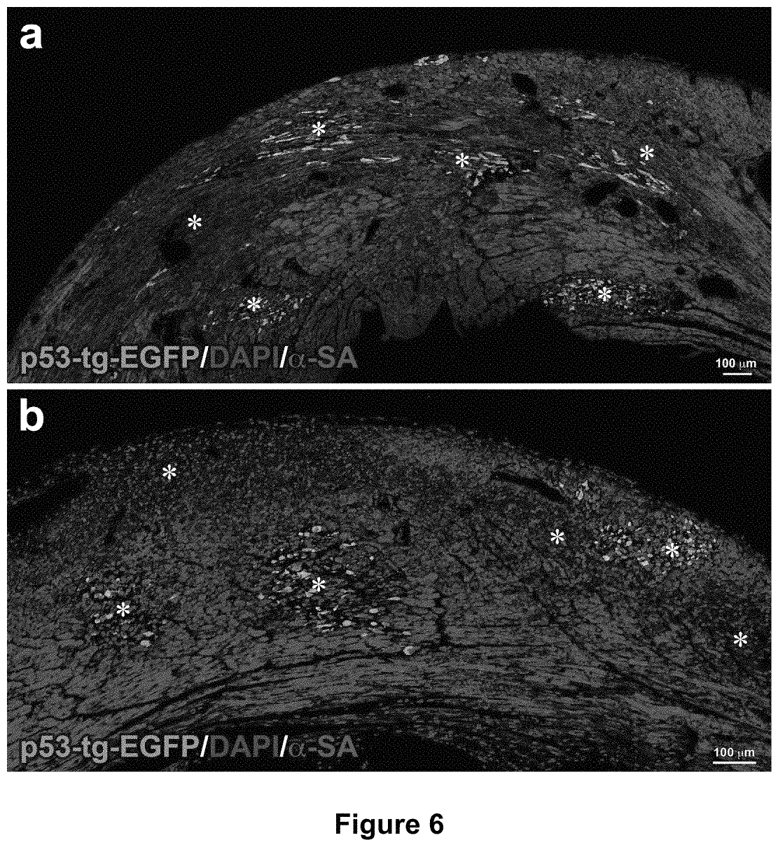

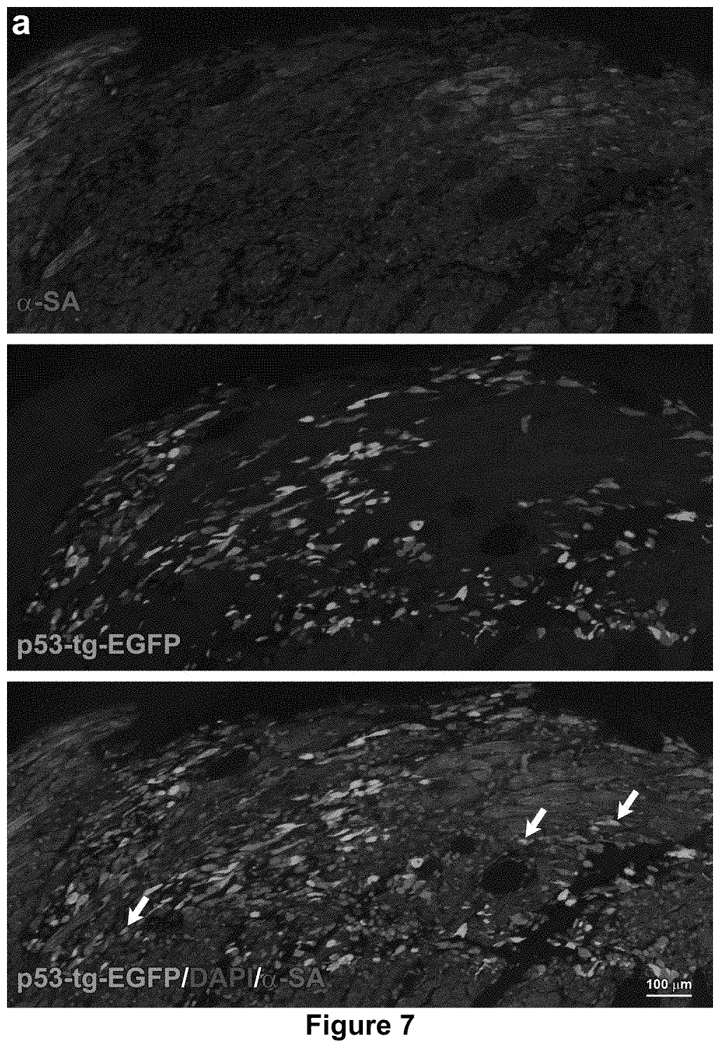

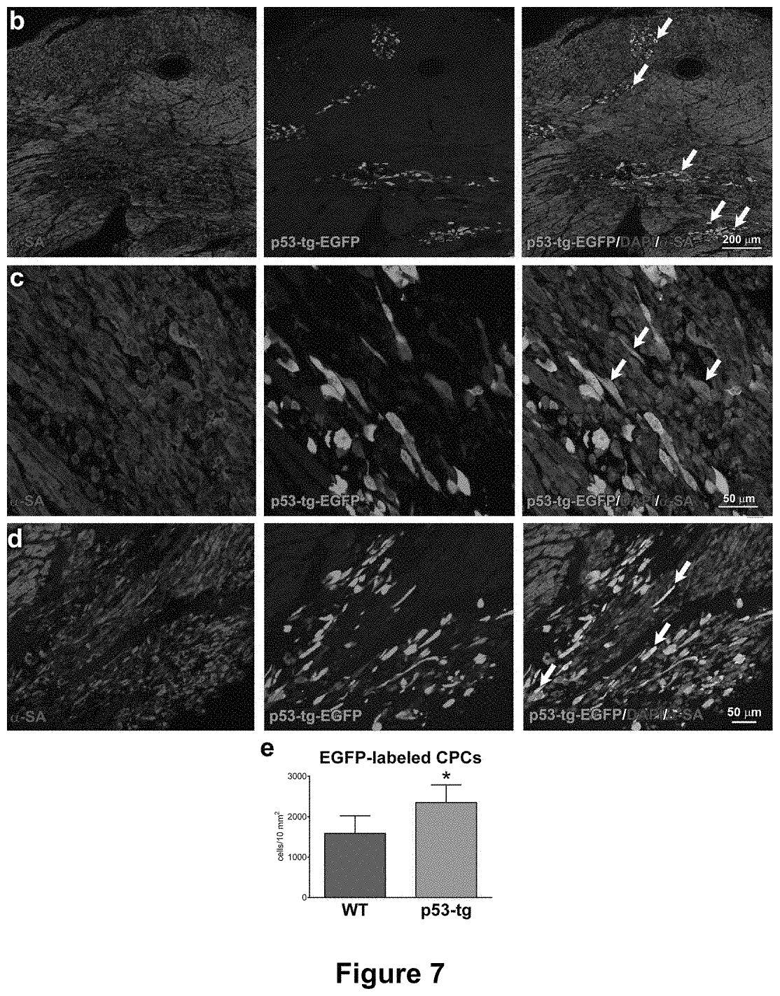

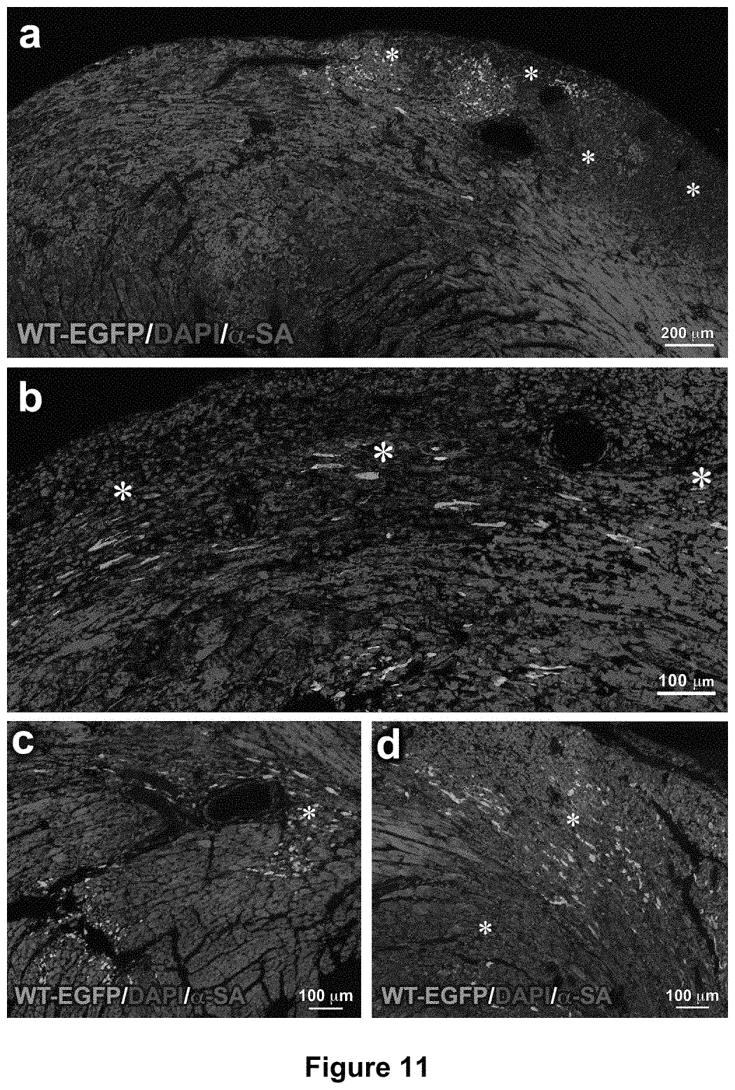

The in vitro results discussed thus far have suggested that p53-tg-CPCs have the capacity to grow extensively in vitro and are more resistant to ROS than WT-CPCs. These two characteristics are critical for the successful implementation of cell therapy for the pathologic heart. Tissue reconstitution involves isolation, in vitro expansion and delivery of CPCs to the damaged myocardium, where the hostile environment and high levels of oxidative stress (Kizil et al., 2015) interfere with the cardiac repair process and cardiomyocyte regeneration (Broughton and Sussman, 2016). To test whether p53-tg-CPCs retained in vivo the properties documented in vitro, both WT-CPCs and p53-tg-CPCs were injected intramyocardially in diabetic mice 3-4 weeks after the administration of streptozotocin when the blood glucose level was >400 mg/dl. This model was selected because is characterized by enhanced oxygen toxicity (Rota et al., 2006). Animals, 4 in each group, were sacrificed 3 days later when engraftment of CPCs is expected to be completed and cell differentiation may begin to occur. This protocol was based on previous observations concerning the engraftment and lineage specification of c-kit-positive hematopoietic stem cells delivered to the damaged myocardium (Rota et al., 2007). Four injections of EGFP-labeled CPCs were performed in different sites of the LV wall. Diabetes was characterized by foci of tissue injury where both WT-CPCs and p53-tg-CPCs homed (FIG. 6; FIG. 11) and began to acquire the cardiomyocyte phenotype (FIGS. 7A-7D). Quantitatively, the number of EGFP-positive cells in the LV myocardium was 2350/10 mm² and 1590/10 mm² in diabetic mice treated with p53-tg-CPCs and WT-CPCs, respectively (FIG. 7E).

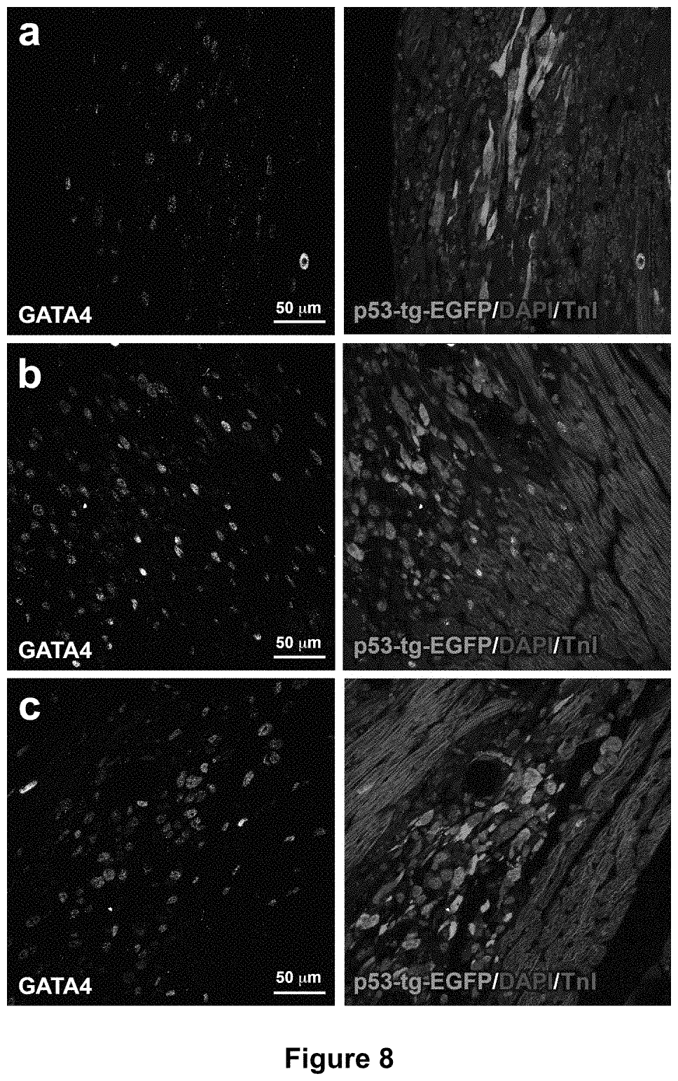

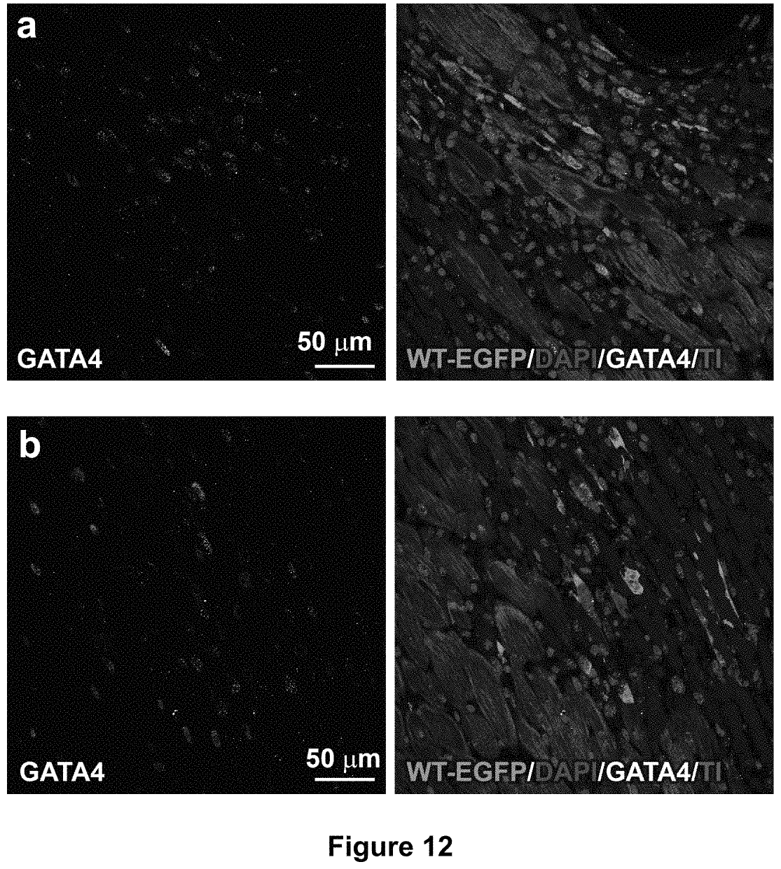

Additionally, clusters of EGFP-positive cells in the early stage of myocyte commitment were recognized by the expression of the transcription factor GATA4 (FIG. 8; FIG. 12). The volume of these developing myocytes can be expected to increase with time and reach in part an adult cell phenotype, as observed previously by in situ activation of endogenous CPCs after myocardial infarction. Importantly, the generation of parenchymal cells in that setting was associated with growth of both resistance arterioles and capillary profiles (Urbanek et al., 2005). Thus, CPCs carrying an extra copy of the p53 gene have an intrinsic advantage and a superior cellular regenerative response after injection in the diabetic heart.

### Example 3: Discussion

The results of the current study indicate that CPCs obtained from the heart of old mice carrying an extra gene-dose of p53 can be propagated extensively in vitro retaining an impressive growth reserve at late passages. Based on this genetic modification, large quantities of CPCs can be generated, raising the possibility that multiple temporally distinct deliveries of cells can be introduced to restore the structural and functional integrity of the damaged myocardium. This critical aspect of autologous cell therapy has recently been documented experimentally (Tokita et al., 2016). Although it might be intuitively obvious that one injection of CPCs cannot reverse cardiac pathology, this work has provided the information needed for the development of a better strategy for the treatment of human heart failure. Thus, a large number of the patient's own CPCs is required, together with the ability of the expanded cells to engraft within the unfavorable environment of the diseased heart.

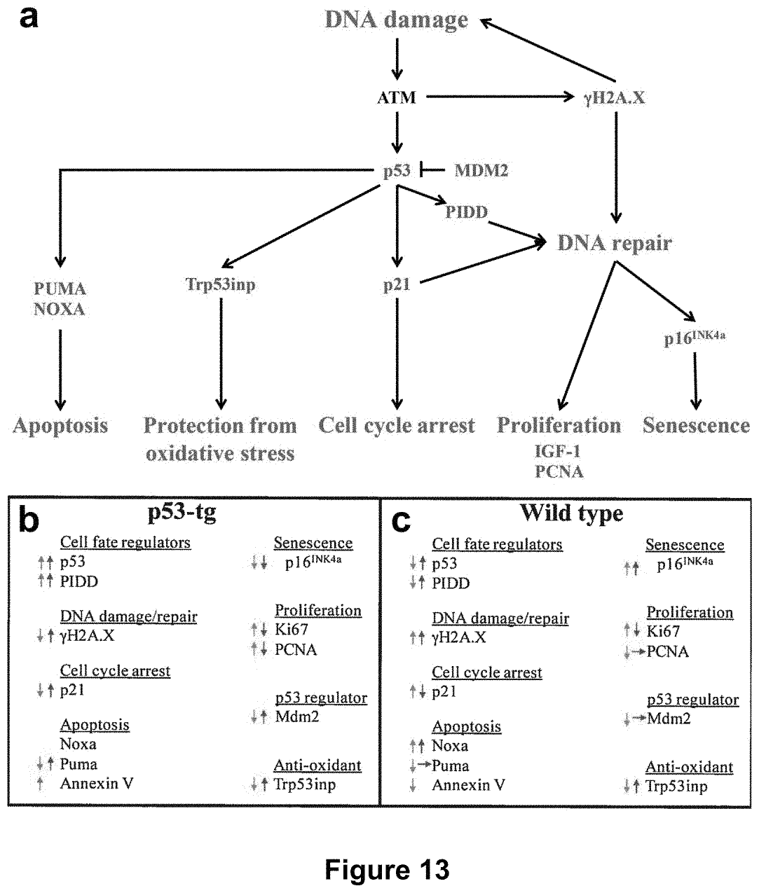

As documented in the current study, the enhanced expression of p53 leads to a complex cellular response which involves a network of genes implicated in DNA repair and cell proliferation, and cellular senescence and apoptosis (FIG. 13). The extra copy of the p53 gene improves the ability of CPCs to sustain oxidative stress, an adaptation mediated by a rapid restoration of the integrity of the DNA and cell division. The prompt and efficient recruitment of DDR proteins at the sites of DNA strand breaks in p53-tg-CPCs reflects the mechanism needed to counteract the consequences of DNA damaging agents, typically present in the diabetic, old and failing heart (Frustaci et al., 2000, Dimmeler and Leri, 2008, Goichberg et al., 2014).

Conversely, CPCs with unmodified quantity of endogenous p53 are less resistant to oxidative stress and fail to mend proficiently DNA strand breaks, a defect that results in irreversible growth arrest and cell death. Thus, p53-tg-CPCs have a significant biological and therapeutic advantage with respect to WT-CPCs; they manifest a higher survival rate when delivered in vivo enhancing cell homing and potentially myocardial regeneration. The increased dosage of p53 provides CPCs with critical defense mechanisms necessary for the cells to remain viable in the adverse milieu of the diabetic and failing heart.

Despite severe hyperglycemia and its toxic consequences, p53-tg-CPCs engraft more effectively than WT-CPCs within the sites of damage present throughout the myocardium of diabetic mice and initiate a reparative process. The difference in the magnitude of cell homing observed with WT-CPCs and p53-tg-CPCs in the presence of diabetes underscores how critical is the function of p53 in enhancing the ability of the delivered cells to colonize the injured ventricle, a condition necessary for the successful replacement of cardiomyocytes lost as a result of cardiac pathology (Leri et al., 2015).

Human CPCs have recently been introduced in the management of heart failure in patients suffering from post-infarction ischemic cardiomyopathy with encouraging results (Chugh et al., 2012, Makkar et al., 2012). However, several clinical trials with a variety of progenitor cells have been performed in the last decade in similar patient cohorts but the outcome has been inconsistent (Afzal et al., 2015). Despite the use of large number of cells, there is general agreement that the fraction of engrafted cells is miniscule and this limitation precludes an efficient recovery of the injured myocardium. The strategy employed here may overcome in part this problem and make stem cell therapy more effective in restoring the structural and functional integrity of the decompensated human heart.

Poor survival and limited retention of adoptively transferred stem cells in the pathologic heart may reduce significantly the efficacy of regenerative therapy. Stem cell viability is influenced by the ischemic condition and inflammatory response of the recipient myocardium and the intrinsic properties of donor cells (Broughton and Sussman, 2016). Several strategies have been utilized to reduce the susceptibility of the delivered cells to die and prolong the window of time available for their engraftment within the damaged myocardium. Preconditioning of CPCs with a variety of cytokines potentiates their resistance to oxidative stress, favoring their migration and recruitment.

A more prolonged effect is obtained when stem cells are genetically modified to express anti-apoptotic mediators. Canonical regulators of myocyte survival, oncogenic proteins and factors involved in the development of embryonic-fetal myocyte progenitors have been employed (Broughton and Sussman, 2016). The serine/threonine Pim-1 kinase which is a downstream target of Akt favors the engraftment and lineage commitment of CPCs and long-term myocardial regeneration (Cottage et al., 2012, Mohsin et al., 2013). CPCs obtained by p53-tg mice show characteristics similar to those observed in the presence of Pim-1: the increased proliferation and delayed cell aging in vitro are accompanied by enhanced engraftment and survival in vivo. The extra gene copy of p53, however, provides an additional advantage through the selective depletion of old damaged stem cells maintaining a pool of progenitors with a younger cell phenotype.

The structural integration of the delivered CPCs with the recipient organ is the primary event that conditions the long-term recovery of the lost myocardium. However, in the current study, we did not evaluate the durability of the process, which will be determined in future work with the expectation that the injected p53-tg-CPCs will differentiate and generate mature, functionally-competent cardiomyocytes, together with the required coronary microcirculation. At the early time point, the injected WT-CPCs and p53-tg-CPCs were restricted to the injured regions of the ventricular wall. The microenvironment of the damaged diabetic myocardium is unquestionably hostile although obligatory for cell homing. The transplantation of progenitor cells in the intact tissue results in cell apoptosis (Tillmanns et al., 2008).

The function of p53 as fate modulator has been studied in several stem cell systems, where it exerts opposite functions, which appear to be context and cell type dependent. p53 orchestrates the polarity of self-renewing divisions in neural stem cells and coordinates the timing for cell fate specification (Quadrato and Di Giovanni, 2012). During steady-state hematopoiesis, the basal-level of p53 activity regulates the quiescence and self-renewal of hematopoietic stem cells (HSCs) expanding the immature cell pool (Liu et al., 2009a). This phenomenon may overcome the decline in HSC function observed with aging, although a larger pool of HSCs with intense self-renewal capacity may favor the development of leukemia (Asai et al., 2011).

The ability of the heart to maintain the steady state and respond to injury declines with aging and diabetes (Eming et al., 2014). The composition of the stem cell pool changes in both cases, favoring the accumulation of cells that do not self-renew and may manifest a skewed pattern of lineage choices. Apoptosis is restricted to p16^{INK4a}-positive CPCs, but the process of clearance of old CPCs is inefficient resulting in their progressive accumulation (Sanada et al., 2014). Enhanced p53 expression corrects the abnormal behavior of CPCs, modifying their fate. As shown here, in the presence of oxidative stress, p53 upregulates the expression of Trp53inp and PIDD in CPCs ameliorating DDR Additionally, p53 increases the level of Puma favoring apoptosis of damaged CPCs. Thus, p53, through cell death activation, prevents the secretory activity of senescent CPCs which release a variety of molecules exerting pro-aging effects on the surrounding young cells (Tchkonia et al., 2013).

Stem cells constitute a long-lived replicative cell population that experiences prolonged periods of quiescence. Stem cell quiescence protects from endogenous stresses mediated by cell respiration and DNA division, but these functions are attenuated by oxidative stress. Old, rarely dividing cells show more γH2AX foci than actively proliferating cells (Rossi et al., 2007, Liu et al., 2009b), since the molecular control of DNA repair is intimately linked to the progression of the cell cycle. Importantly, the extent of DNA damage is comparable in WT-CPCs and p53-tg-CPCs but the enhanced expression of p53 expands the pool of cells displaying DDR foci. This biological response supports the view that CPCs genetically modified to express physiologically regulated p53 are protected from environmental stimuli and genomic lesions. DNA repair maintains genomic integrity and attenuates the rate of aging of p53-tg-CPCs.

Whether the enhanced expression of p53 improves the intrinsic properties of CPCs, or the intact resident stem cell compartment is activated by the intramyocardial injection of specific growth factors, these cells are responsible for myocyte and coronary vessel regeneration (Beltrami et al., 2003, Sanada et al., 2014, Liu et al., 2015). The replicative reserve of c-kit-positive CPCs predicts the evolution of ischemic cardiomyopathy following revascularization in humans (D'Amario et al., 2014) and profound defects in human CPC function are present with advanced heart failure (Urbanek et al., 2003, Urbanek et al., 2005) and in the decompensated senescent human heart (Chimenti et al., 2003). CPCs are the critical determinant of human cardiac pathology and strategies increasing their growth and reparative process may have important clinical implications.

### REFERENCES

Afzal, M. R., Samanta, A., Shah, Z. I., Jeevanantham, V., Abdel-Latif, A., Zuba-Surma, E. K., and Dawn, B. Adult bone marrow cell therapy for ischemic heart disease: evidence and insights from randomized controlled trials. Circ. Res. 2015; 117: 558-575
Asai, T., Liu, Y., Bae, N., and Nimer, S. D. The p53 tumor suppressor protein regulates hematopoietic stem cell fate. J. Cell. Physiol. 2011: 226: 2215-2221
Beausejour, C. M. and Campisi, J. Ageing: balancing regeneration and cancer. Nature. 2006; 443: 404-405
Beltrami, A. P., Barlucchi, L., Torella, D., Baker, M., Limana, F., Chimenti, S., Kasahara, H., Rota, M., Musso, E., Urbanek, K. et al. Adult cardiac stem cells are multipotent and support myocardial regeneration. Cell. 2003; 114: 763-776
Bock, F. J., Peintner, L., Tanzer, M., Manzl, C., and Villunger, A. P53-induced protein with a death domain (PIDD): master of puppets?. Oncogene. 2012; 31: 4733-4739
Broughton, K. M. and Sussman, M. A. Empowering adult stem cells for myocardial regeneration v2.0: success in small steps. Circ. Res. 2016; 118: 867-880
Cheng, Z., Ito, S., Nishio, N., Thanasegaran, S., Fang, H., and Isobe, K. Characteristics of cardiac aging in C57BL/6 mice. Exp. Gerontol. 2013; 48: 341-348
Chimenti, C., Kajstura, J., Torella, D., Urbanek, K., Heleniak, H., Colussi, C., Di Meglio, F., Nadal-Ginard, B., Frustaci, A., Leri, A. et al. Senescence and death of primitive cells and myocytes lead to premature cardiac aging and heart failure. Circ. Res. 2003; 93: 604-613
Chugh, A. R, Beache, G. M., Loughran, J. H., Mewton, N., Elmore, J. B., Kajstura, J., Pappas, P., Tatooles, A., Stoddard, M. F., Lima, J. A. et al. Administration of cardiac stem cells in patients with ischemic cardiomyopathy: the SCIPIO trial: surgical aspects and interim analysis of myocardial function and viability by magnetic resonance. Circulation. 2012; 126: S54-S64
Cottage, C. T., Neidig, L., Sundararaman, B., Din, S., Joyo, A. Y., Bailey, B., Gude, N., Hariharan, N., and Sussman, M. A. Increased mitotic rate coincident with transient telomere lengthening resulting from pim-1 overexpression in cardiac progenitor cells. Stem Cells. 2012; 30: 2512-2522
D'Amario, D., Fiorini, C., Campbell, P. M., Goichberg, P., Sanada, F., Zheng, H., Hosoda, T., Rota, M., Connell, J. M. et al. Functionally competent cardiac stem cells can be isolated from endomyocardial biopsies of patients with advanced cardiomyopathies. Circ. Res. 2011; 108: 857-861
D'Amario, D., Leone, A. M., Iaconelli, A., Luciani, M., Gaudino, M., Kannappan, R, Manchi, M., Severino, A., Shin, S. H., Graziani, F. et al. Growth properties of cardiac stem cells are a novel biomarker of patients' outcome after coronary bypass surgery. Circulation. 2014; 129: 157-172
Dimmeler, S. and Leri, A. Aging and disease as modifiers of efficacy of cell therapy. Circ. Res. 2008; 102: 1319-1330
Eming, S. A., Martin, P., and Tomic-Canic, M. Wound repair and regeneration: mechanisms, signaling, and translation. (265sr6) Sci. Transl. Med. 2014; 6
Frustaci, A., Kajstura, J., Chimenti, C., Jakoniuk, I., Leri, A., Maseri, A., Nadal-Ginard, B., and Anversa, P. Myocardial cell death in human diabetes. Circ. Res. 2000; 87: 1123-1132
Fumagalli, M., Rossiello, F., Clerici, M., Barozzi, S., Cittaro, D., Kaplunov, J. M., Bucci, G., Dobreva, M., Matti, V., Beausejour, C. M. et al. Telomeric DNA damage is irreparable and causes persistent DNA-damage-response activation. Nat. Cell Biol. 2012; 14: 355-365
Garcia-Cao, I., Garcia-Cao, M, Martin-Caballero, J., Criado, L. M., Klatt, P., Flores, J. M., Weill, J. C., Blasco, M. A., and Serrano, M. “Super p53” mice exhibit enhanced DNA damage response, are tumor resistant and age normally. EMBO J. 2002; 21: 6225-6235
Garcia-Cao, I., Garcia-Cao, M, Tomas-Loba, A., Martin-Caballero, J., Flores, J. M., Klatt, P., Blasco, M. A., and Serrano, M. Increased p53 activity does not accelerate telomere-driven ageing. EMBO Rep. 2006; 7: 546-552
Goichberg, P., Kannappan, R., Cimini, M., Bai, Y., Sanada, F., Sorrentino, A., Signore, S., Kajstura, J., Rota, M., Anversa, P. et al. Age-associated defects in EphA2 signaling impair the migration of human cardiac progenitor cells. Circulation. 2013; 128: 2211-2223
Goichberg, P., Chang, J., Liao, R. and Leri, A. Cardiac stem cells: biology and clinical applications. Antioxid. Redox Signal. 2014; 21: 2002-2017
Hachamovitch, R., Wicker, P., Capasso, J. M., and Anversa, P. Alterations of coronary blood flow and reserve with aging in Fischer 344 rats. Am. J. Phys. 1989; 256: H66-H73
Harris, V. M. Protein detection by simple Western analysis. Methods Mol. Biol. 2015; 1312: 465-468
Hosoda, T., D'Amario, D., Cabral-Da-Silva, M. C., Zheng, H., Padin-Iruegas, M. E., Ogorek, B., Ferreira-Martins, J., Yasuzawa-Amano, S., Amano, K., and Ide-Iwata, N. Clonality of mouse and human cardiomyogenesis in vivo. Proc. Natl. Acad. Sci. U.S.A 2009; 106: 17169-17174
Kizil, C., Kyritsis, N., and Brand, M. Effects of inflammation on stem cells: together they strive?. EMBO Rep. 2015; 16: 416-426
Leri, A., Claudio, P. P., Li, Q., Wang, X., Reiss, K., Wang, S., Malhotra, A., Kajstura, J., and Anversa, P. Stretch-mediated release of angiotensin 11 induces myocyte apoptosis by activating p53 that enhances the local renin-angiotensin system and decreases the Bcl-2-to-Bax protein ratio in the cell. J. Clin. Invest. 1998; 101: 1326-1342
Leri, A., Liu, Y., Wang, X., Kajstura, J., Malhotra, A., Meggs, L. G., and Anversa, P. Overexpression of insulin-like growth factor-1 attenuates the myocyte renin-angiotensin system in transgenic mice. Circ. Res. 1999; 84: 752-762
Leri, A., Franco, S., Zacheo, A., Barlucchi, L., Chimenti, S., Limana, F., Nadal-Ginard, B., Kajstura, J., Anversa, P., and Blasco, M. A. Ablation of telomerase and telomere loss leads to cardiac dilatation and heart failure associated with p53 upregulation. EMBO J. 2003; 22: 131-139
Leri, A., Rota, M., Pasqualini, F. S., Goichberg, P., and Anversa, P. Origin of cardiomyocytes in the adult heart. Circ. Res. 2015; 116: 150-166
Liu, Y., Elf, S. E., Miyata, Y., Sashida, G., Liu, Y., Huang, G., Di Giandomenico, S., Lee, J. M., Deblasio, A., Menendez, S. et al. p53 regulates hematopoietic stem cell quiescence. Cell Stem Cell. 2009; 4: 37-48
Liu, Y., Elf, S. E., Asai, T., Miyata, Y., Liu, Y., Sashida, G., Huang, G., Di Giandomenico, S., Koff, A., and Nimer, S. D. The p53 tumor suppressor protein is a critical regulator of hematopoietic stem cell behavior. Cell Cycle. 2009; 8: 3120-3124
Liu, X., Hall, S. R, Wang, Z., Huang, H., Ghanta, S., Di Sante, M., Leri, A., Anversa, P., and Perrella, M. Rescue of neonatal cardiac dysfunction in mice by administration of cardiac progenitor cells in utero. Nat. Commun. 2015; 6: 8825
Lorenzo, Y., Costa, S., Collins, A. R., and Azqueta, A. The comet assay, DNA damage, DNA repair and cytotoxicity: hedgehogs are not always dead. Mutagenesis. 2013; 28: 427-432
Loughery, J. and Meek, D. Switching on p53: an essential role for protein phosphorylation?. BioDiscovery. 2013; 8: 1
Lukas, J., Lukas, C., and Bartek, J. More than just a focus: the chromatin response to DNA damage and its role in genome integrity maintenance. Nat. Cell Biol. 2011; 13: 1161-1169
Makkar, R. R., Smith, R. R., Cheng, K., Malliaras, K., Thomson, L. E., Berman, D., Czer, L. S., Marban, L., Mendizabal, A., Johnston, P. V. et al. Intracoronary cardiosphere-derived cells for heart regeneration after myocardial infarction (CADUCEUS): a prospective, randomised phase I trial. Lancet. 2012; 379: 895-904
Matheu, A., Maraver, A., Klatt, P., Flores, I., Garcia-Cao, I., Borras, C., Flores, J. M., Villa, J., Blasco, M. A., and Serrano, M. Delayed ageing through damage protection by the Arf/p53 pathway. Nature. 2007; 448: 375-379
Matheu, A., Maraver, A., and Serrano, M. The Arf/p53 pathway in cancer and aging. Cancer Res. 2008; 68: 6031-6034
McDonald, J. H. Handbook of Biological Statistics. third ed. Sparky House Publishing, Baltimore, Md.; 2014
Mohrin, M., Bourke, E., Alexander, D., Warr, M. R., Barry-Holson, K., Le Beau, M. M., Morrison, C. G., and Passegue, E. Hematopoietic stem cell quiescence promotes error-prone DNA repair and mutagenesis. Cell Stem Cell. 2010; 7: 174-185
Mohsin, S., Khan, M., Nguyen, J., Alkatib, M., Siddiqi, S., Hariharan, N., Wallach, K., Monsanto, M., Gude, N., Dembitsky, W. et al. Rejuvenation of human cardiac progenitor cells with Pim-1 kinase. Circ. Res. 2013; 113: 1169-1179
Quadrato, G. and Di Giovanni, S. Gatekeeper between quiescence and differentiation: p53 in axonal outgrowth and neurogenesis. Int. Rev. Neurobiol. 2012; 105: 71-89
Riley, T., Sontag, E., Chen, P., and Levine, A. Transcriptional control of human p53-regulated genes. Nat. Rev. Mol. Cell. Biol. 2008; 9: 402-412
Rossi, D. J., Seita, J., Czechowicz, A., Bhattacharya, D., Bryder, D., and Weissman, I. L. Hematopoietic stem cell quiescence attenuates DNA damage response and permits DNA damage accumulation during aging. Cell Cycle. 2007; 6: 2371-2376
Rota, M., LeCapitaine, N., Hosoda, T., Boni, A., De Angelis, A., Padin-Iruegas, M. E., Esposito, G., Vitale, S., Urbanek, K., Casarsa, C. et al. Diabetes promotes cardiac stem cell aging and heart failure, which are prevented by deletion of the p66shc gene. Circ. Res. 2006; 99: 42-52
Rota, M., Kajstura, J., Hosoda, T., Bearzi, C., Vitale, S., Esposito, G., Iaffaldano, G., Padin-Iruegas, M. E., Gonzalez, A., Rizzi, R. et al. Bone marrow cells adopt the cardiomyogenic fate in vivo. Proc. Natl. Acad. Sci. U.S.A 2007; 104: 17783-17788
Sanada, F., Kim, J., Czarna, A., Chan, N. Y., Signore, S., Ogorek, B., Isobe, K., Wybieralska, E., Borghetti, G., Pesapane, A. et al. c-kit-positive cardiac stem cells nested in hypoxic niches are activated by stem cell factor reversing the aging myopathy. Circ. Res. 2014; 114: 41-55
Schieber, M. and Chandel, N. S. ROS function in redox signaling and oxidative stress. Curr. Biol. 2014; 24: R453-R462
Serrano, M. and Blasco, M. A. Cancer and ageing: convergent and divergent mechanisms. Nat. Rev. Mol. Cell. Biol. 2007; 8: 715-722
Signore, S., Sorrentino, A., Borghetti, G., Cannata, A., Meo, M., Zhou, Y., Kannappan, R., Pasqualini, F., O'Malley, H., Sundman, M. et al. Late Na (+) current and protracted electrical recovery are critical determinants of the aging myopathy. Nat. Commun. 2015; 6: 8803
Tchkonia, T., Zhu, Y., van Deursen, J., Campisi, J., and Kirkland, J. L. Cellular senescence and the senescent secretory phenotype: therapeutic opportunities. J. Clin. Invest. 2013; 123: 966-972
Tillmanns, J., Rota, M., Hosoda, T., Misao, Y., Esposito, G., Gonzalez, A., Vitale, S., Parolin, C., Yasuzawa-Amano, S., Muraski, J. et al. Formation of large coronary arteries by cardiac progenitor cells. Proc. Natl. Acad. Sci. U.S.A 2008; 105: 1668-1673
Tokita, Y., Tang, X. L., Li, Q., Wysoczynski, M., Hong, K. U., Nakamura, S., Wu, W. J., Xie, W., Li, D., Hunt, G. et al. Repeated administrations of cardiac progenitor cells are markedly more effective than a single administration: a new paradigm in cell therapy. Circ. Res. 2016; 119: 635-651
Torella, D., Rota, M., Nurzynska, D., Musso, E., Monsen, A., Shiraishi, I., Zias, E., Walsh, K., Rosenzweig, A., Sussman, M. A. et al. Cardiac stem cell and myocyte aging, heart failure, and insulin-like growth factor-1 overexpression. Circ. Res. 2004; 94: 514-524
Urbanek, K., Quaini, F., Tasca, G., Torella, D., Castaldo, C., Nadal-Ginard, B., Leri, A., Kajstura, J., Quaini, E., and Anversa, P. Intense myocyte formation from cardiac stem cells in human cardiac hypertrophy. Proc. Natl. Acad. Sci. U.S.A 2003; 100: 10440-10445
Urbanek, K., Torella, D., Sheikh, F., De Angelis, A., Nurzynska, D., Silvestri, F., Beltrami, C. A., Bussani, R, Beltrami, A. P., Quaini, F. et al. Myocardial regeneration by activation of multipotent cardiac stem cells in ischemic heart failure. Proc. Natl. Acad. Sci. U.S.A 2005; 102: 8692-8697
Valente, L. J., Gray, D. H., Michalak, E. M., Pinon-Hofbauer, J., Egle, A., Scott, C. L., Janic, A., and Strasser, A. p53 efficiently suppresses tumor development in the complete absence of its cell-cycle inhibitory and proapoptotic effectors p21, Puma, and Noxa. Cell Rep. 2013; 3: 1339-1345
Xu, J., Carretero, O. A., Liao, T. D., Peng, H., Shesely, E. G., Xu, J., Liu, T. S., Yang, J. J., Reudelhuber, T. L., and Yang, X. P. Local angiotensin II aggravates cardiac remodeling in hypertension. Am. J. Physiol. Heart Circ. Physiol. 2010; 299: H1328-H1338
Xu, C., Zhang, L., Duan, L., and Lu, C. MicroRNA-3196 is inhibited by H2AX phosphorylation and attenuates lung cancer cell apoptosis by downregulating PUMA. Oncotarget. 2016; 7: 77764-77776

1. A method of treating or preventing a heart disease or disorder in a subject in need thereof comprising administering isolated cardiac progenitor cells (CPCs) to the subject, wherein the CPCs comprise one or more copies of a tumor suppressor p53 gene in addition to the endogenous copy of a p53 gene.

2. The method of claim 1, wherein the heart disease or disorder is heart failure, diabetic heart disease, rheumatic heart disease, hypertensive heart disease, ischemic heart disease, cerebrovascular heart disease, inflammatory heart disease and/or congenital heart disease.

3. The method of claim 1, wherein the CPCs express an increased amount of p53 protein compared to the amount expressed by CPCs that do not comprise one or more copies of a p53 gene in addition to the endogenous copy of a p53 gene.

4. A method of repairing and/or regenerating damaged tissue of a heart in a subject in need thereof comprising:
(a) extracting cardiac progenitor cells (CPCs) from a heart;
(b) introducing one or more tumor suppressor p53 genes into the CPCs of step (a);
(c) culturing and expanding said CPCs from step (b); and
(d) administering a dose of said CPCs from step (c) to an area of damaged tissue in the subject.

5. The method of claim 4, wherein the dose of said CPCs administered to the area of damaged tissue in the subject is effective to (i) repair and/or regenerate the damaged tissue of the heart, and/or (ii) to promote cellular engraftment and growth of the CPCs in the damaged tissue of the heart in a subject in need thereof.

6. The method of claim 4, wherein the subject has diabetes.

7. A method of producing a large quantity of cardiac progenitor cells (CPCs) comprising:
(a) isolating CPCs from heart tissue;
(b) introducing one or more tumor suppressor p53 genes into the CPCs of step (a); and
(c) culturing and expanding the CPCs of step (b), thereby
(i) producing a large quantity of CPCs,
(ii) producing CPCs having an improved ability to tolerate oxidative stress compared to CPC's from step (a),
(iii) producing CPCs having restored DNA integrity compared to CPCs from step (a), and/or
(iv) producing CPCs having an improved proliferative capacity compared to CPCs from step (a).

8. A method of promoting cellular engraftment and growth of cells in an organ or tissue during cell therapy, comprising:
(a) extracting cells from an organ or tissue;
(b) introducing one or more tumor suppressor p53 genes into the cells of step (a);
(c) culturing and expanding said cells from step (b); and
(d) applying an amount of said cells from step (c) to an area of damaged organ or tissue, thereby promoting cellular engraftment and growth of cells in the damaged organ or tissue.

9. The method of claim 7, wherein culturing and expanding the CPCs of step (b) thereby produces
CPCs having an improved ability to tolerate oxidative stress compared to CPCs from step (a).

10. The method of claim 7, wherein culturing and expanding the CPCs of step (b) thereby produces
CPCs having restored DNA integrity compared to CPCs from step (a).

11. The method of claim 7, wherein culturing and expanding the CPCs of step (b) thereby produces
CPCs having an improved proliferative capacity compared to CPCs from step (a).

12. A pharmaceutical composition comprising a therapeutically effective amount of isolated cardiac progenitor cells (CPCs) and a pharmaceutically acceptable carrier, wherein said isolated CPCs comprise one or more copies of a tumor suppressor p53 gene in addition to the endogenous copy of a p53 gene.

13. The pharmaceutical composition of claim 12, wherein the pharmaceutically acceptable carrier is for (i) repairing and/or regenerating damaged tissue of a heart, or (ii) promoting cellular engraftment and growth of the CPCs in damaged tissue of a heart.

14. A pharmaceutical composition comprising a therapeutically effective amount of cells and a pharmaceutically acceptable carrier for promoting cellular engraftment and growth of the cells in a damaged organ or tissue, wherein said cells comprise one or more copies of a tumor suppressor p53 gene in addition to the endogenous copy of a p53 gene.

15. The method of claim 5, wherein the subject has diabetes.

16. The method of claim 7, wherein culturing and expanding the CPCs of step (b) thereby produces a large quantity of CPCs.


SEQUENCE LISTING (24 sequences)

SEQ ID NO: 1 - DNA, 20 bp, Mus sp.
cgtgaacatgttgttgaggc

SEQ ID NO: 2 - DNA, 20 bp, Mus sp.
gcagaagagctgctacgtga

SEQ ID NO: 3 - DNA, 20 bp, Mus sp.
tggatgctcttcagttcgtg

SEQ ID NO: 4 - DNA, 20 bp, Mus sp.
cactcatccacaatgcctgt

SEQ ID NO: 5 - DNA, 20 bp, Mus sp.
ggtcagagagacgcttaccg

SEQ ID NO: 6 - DNA, 20 bp, Mus sp.
gtagttgagtcgctggggaa

SEQ ID NO: 7 - DNA, 20 bp, Mus sp.
ccaggattggacatggtgcc

SEQ ID NO: 8 - DNA, 22 bp, Mus sp.
gtgaggaggagcatgaatggag

SEQ ID NO: 9 - DNA, 18 bp, Mus sp.
cgggctagaccctctacg

SEQ ID NO: 10 - DNA, 18 bp, Mus sp.
agccctccagaaggcaac

SEQ ID NO: 11 - DNA, 20 bp, Mus sp.
ttcaagtctgctggcacccg

SEQ ID NO: 12 - DNA, 20 bp, Mus sp.
aacgcgccagtgaacccaac

SEQ ID NO: 13 - DNA, 20 bp, Mus sp.
ctagcattcaggccctcatc

SEQ ID NO: 14 - DNA, 20 bp, Mus sp.
tccgactgtgactcctccat

SEQ ID NO: 15 - DNA, 20 bp, Mus sp.
tggataaagaagaggaggcg

SEQ ID NO: 16 - DNA, 20 bp, Mus sp.
ggagacagtggagtggcttt

SEQ ID NO: 17 - DNA, 20 bp, Mus sp.
aaggttccgtggagtctgct

SEQ ID NO: 18 - DNA, 20 bp, Mus sp.
cagagtggtcagggttccat

SEQ ID NO: 19 - DNA, 18 bp, Mus sp.
ctacctcagcacccgcag

SEQ ID NO: 20 - DNA, 20 bp, Mus sp.
gcccaatatcacagacgaga

SEQ ID NO: 21 - DNA, 20 bp, Mus sp.
tctgtgaaggagcacaggaa

SEQ ID NO: 22 - DNA, 20 bp, Mus sp.
ctgctctcactcagcgatgt

SEQ ID NO: 23 - DNA, 19 bp, Mus sp.
atgtgaggcgggtggaacg

SEQ ID NO: 24 - DNA, 21 bp, Mus sp.
ctcggtgaccctggtcttttg